(19) United States
(12) Patent Application Publication
(10) Pub. No.: US 2015/0126621 A1
(43) Pub. Date: May 7, 2015

(54) METHOD FOR PREDICTING RECURRENCE OF MELANOMA USING miRNA ALTERATIONS

(71) Applicant: New York University, New York, NY (US)

(72) Inventors: Douglas Hanniford, New York, NY (US); Eva Hernando-Monge, New York, NY (US); Iman Osman, Jersey City, NJ (US); Shulian Shang, Elmhurst, NY (US); Yongzhao Shao, Forest Hills, NY (US)

(73) Assignee: New York University, New York, NY (US)

(21) Appl. No.: 14/400,830

(22) PCT Filed: May 15, 2013
(86) PCT No.: PCT/US13/41216

§ 371 Date: Nov. 13, 2014

Related U.S. Application Data
(60) Provisional application No. 61/647,471, filed on May 15, 2012.

(51) Int. Cl. C12Q 1/68

(52) U.S. Cl. CPC C12Q 1/6886; C12Q 2600/178; C12Q 2600/118
USPC 514/789; 506/9; 506/16

(57) ABSTRACT

Described herein are miRNA-based methods for prognosis of recurrence of melanoma and related methods and kits. The present invention addresses these and other needs by providing a method for predicting the likelihood of recurrence of melanoma (including distal metastasis and locoregional recurrence) in a subject diagnosed with melanoma. In a separate aspect, the invention provides a method for treatment of a melanoma recurrence (including distal metastasis and locoregional recurrence) in a subject in need thereof.

### CROSS REFERENCE TO RELATED APPLICATIONS

This application claims priority from U.S. Provisional Application Ser. No. 61/647,471, filed on May 15, 2012, which is incorporated herein by reference in its entirety.

### STATEMENT AS TO FEDERALLY SPONSORED RESEARCH

This invention was funded, in part, by Department of Defense (DOD) Collaborative Award CA093471. Accordingly, the U.S. government has certain rights to this invention.

### SEQUENCE LISTING

The instant application contains a Sequence Listing which has been submitted in ASCII format via EFS-Web and is hereby incorporated by reference in its entirety. Said ASCII copy, created on May 8, 2013, is named 243735.000020_SL.txt and is 4,431 bytes in size.

### TECHNICAL FIELD OF THE INVENTION

The invention relates to miRNA-based methods for prognosis of recurrence of melanoma and related methods and kits.

### BACKGROUND OF THE INVENTION

Melanoma originates from uncontrolled proliferation of specialized melanocytes normally responsible for producing pigments in the epithelial layer. Though typically associated with the skin, these cells can also be present in the eye, bone and heart^{1, 2, 3}, and cancer lesions can develop in any of these locations.

Staging of the melanoma at the time of diagnosis incorporates thickness, mitotic index, ulceration, and sentinel lymph node status, and is generally indicative of clinical outcome⁴⁻⁸. Melanoma is curable for most patients whose primary tumors are adequately removed; however, many patients recur and progress to advanced disease and death. The vast majority of recurrent patients present with metastatic disease, from which they eventually succumb. Accordingly, 7.1% and 32.8% of stage I and II patients suffer disease recurrence, respectively⁹.

Clinical staging incorporates information about the primary tumor, regional lymphatics, and distant metastatic sites into the AJCC (American Joint Committee on Cancer) TNM staging system and is the primary means of assessing prognosis¹⁰. It is however insufficient to account for within-stage heterogeneity of disease outcome. Thickness remains the most robust predictor of survival in localized melanoma, but the morphologically-based staging system only partly explains the variability in the natural history of melanoma. Although the AJCC has incorporated the mitotic index into the staging criteria, the biomarkers are limited by inter-observer variability and lack of standardization and hence, have not been integrated into clinical practice¹¹⁻¹³.

Advanced malignant melanoma remains a disease with poor prognosis, with median survival of 8.5 months and a 5-year survival rate of less than 5%¹⁵. This is likely a reflection of the absence of effective treatment for late stage melanoma and hence, the early identification of patients at highest risk for the development of aggressive disease is critical. The identification of biomarkers to aid in the diagnosis and prognosis of the cancer would impact mortality from melanoma¹⁶.

The classic tumorigenesis model posits that stepwise accumulation of genetic changes gradually results in the acquisition of metastatic potential by tumor cells. Recurrence and metastasis occur from primary melanomas that frequently are histologically equivalent to non-recurrent lesions at the time of diagnosis and excision, suggesting that genomic or epigenetic alterations that predetermine a tumor's potential to spread may be acquired early in tumor progression. Evidence has been accumulating in support of such a deterministic model of tumor evolution: mRNA expression profiling in breast and prostate carcinoma have shown that expression profiles of paired primary and metastatic tumors were more similar to each other than to other patient tumors¹⁷. Further, gene expression signatures identified in primary prostate or breast tumors have been predictive of disease recurrence or progression to metastasis¹⁸⁻²⁰.

A recent study identified pro-metastatic genes in melanoma, which are recurrently amplified in primary tumor and also act as classic oncogenes, suggesting that molecular events involved in tumor initiation can dictate clinical outcome²¹. Further, a sequencing study of a primary acral melanoma and its metastasis found that the majority of genetic alterations present in the metastasis were detectable in the primary tumor²².

There is an unmet need in the art for treatment of recurrent melanoma. Identifying molecular alterations that can be measured at the time of melanoma diagnosis that are predictive of disease recurrence would be clinically useful for developing individualized treatment plans and/or to uncover novel therapeutic targets.

The alteration of miRNA expression correlates with cancer progression, and the perturbation of individual miRNAs can functionally impact cancer cell metastasis²⁴⁻³⁰. A study by inventors and co-workers identified several miRNAs that were prognostic in metastatic melanoma and were also found to be altered in primary tumors²³ suggesting that melanoma metastasis may not strictly be a consequence of stepwise accumulation of molecular alterations resulting in rare cells that gain metastatic capacity. Rather, a larger population(s) of cells with metastasis-initiating events may be present at early stages of melanomagenesis.

### SUMMARY OF THE INVENTION

As follows from the Background section, above, there is an unmet need in the art for compositions and methods for prognosis and treatment of recurrent melanoma.

The present invention addresses these and other needs by providing a method for predicting the likelihood of recurrence of melanoma (including distal metastasis and locoregional recurrence) in a subject diagnosed with melanoma, said method comprising:
a. measuring the levels of four or more miRNAs selected from the group consisting of miR-10a, miR-1285, miR-377*, miR-513b, miR-342-3p, miR-625*, SNORD3A, miR-1204, miR-574-3p, let-7a-2*, miR-615-3p, miR-564, miR-154*, miR-663, miR-99b, miR-1276, miR-215, miR-374b*, miR-382, miR-516b, and miR-7, in a melanoma sample collected from the subject;
b. calculating combined levels of the miRNAs measured in step (a);
c. comparing the combined levels of the miRNAs measured in step (a) with the corresponding combined control levels of said miRNAs, and
d. (i) identifying the subject as being at high risk of melanoma recurrence if the combined levels of the miRNAs measured in step (a) are higher than the corresponding combined control levels or (ii) identifying the subject as being at low risk of melanoma recurrence if the combined levels of the miRNAs measured in step (a) are same or lower than the corresponding combined control levels.

In one specific embodiment, the above method comprises measuring the level of miR-10a, miR-1285, miR-374b*, miR-377*, miR-513b, miR-342-3p, miR-625*, SNORD3A, miR-1204, miR-574-3p, let-7a-2*, miR-615-3p, miR-564, miR-154*, miR-7, miR-215, miR-382, miR-663, miR-516b, miR-99b, and miR-1276. In another specific embodiment, the above method comprises measuring the level of miR-374b*, miR-377*, miR-1285, and miR-1276. In yet another specific embodiment, the above method comprises measuring the level of miR-374b*, miR-377*, miR-1285, and miR-1204. In a further specific embodiment, the above method comprises measuring the level of miR-382, miR-1276, and miR-615-3p. In another specific embodiment, the above method comprises measuring the level of miR-215, miR-374b*, miR-382, miR-516b, and miR-7. In yet another specific embodiment, the above method comprises measuring the level of miR-382, miR-516b, and miR-7.

The combined control levels used in the above method can be any suitable control (e.g., a predetermined standard or the combined levels of the same miRNAs in a non-recurrent melanoma sample [e.g., determined by the statistical measure, Youden's Index of the Receiving Operative Characteristic (ROC) curve, see, e.g., Zhou et al. (2011) Statistical Methods in Diagnostic Medicine, 2^{nd} Edition, Wiley, N.J.]).

In one specific embodiment, the subject is human. In another specific embodiment, the subject is an experimental animal.

In one embodiment, the above method comprises a step of collecting the melanoma sample from the subject.

In the above method, the levels of the miRNAs can be determined using any method known in the art (e.g., hybridization [e.g., to miRNA arrays], RT-PCR, sequencing, etc.). In one embodiment, prior to measuring miRNA level, the miRNA is purified from the melanoma sample. In another embodiment, the method of the invention further comprises the step of reducing or eliminating degradation of the miRNA.

In one embodiment, the above method is followed by administering to the subject determined as being at high risk of melanoma recurrence a melanoma treatment. Any melanoma treatment can be used. Non-limiting examples of treatments include, e.g., Interleukin 2 (IL2), Aldesleukin (Proleukin), Dacarbazine (DTIC-Dome), Ipilimumab (Yervoy), temozolomide, Vemurafenib (Zelboraf), and any combinations thereof.

In another embodiment, the above method is followed by recruiting the subject in a clinical trial.

In conjunction with the above prognostic method, the invention provides a kit comprising primers or probes specific for four or more miRNAs selected from the group consisting of miR-10a, miR-1285, miR-374b*, miR-377*, miR-513b, miR-342-3p, miR-625*, SNORD3A, miR-1204, miR-574-3p, let-7a-2*, miR-615-3p, miR-564, miR-154*, miR-7, miR-215, miR-382, miR-663, miR-516b, miR-99b, and miR-1276.

In one specific embodiment, such kit comprises primers or probes specific for miR-10a, miR-1285, miR-374b*, miR-377*, miR-513b, miR-342-3p, miR-625*, SNORD3A, miR-1204, miR-574-3p, let-7a-2*, miR-615-3p, miR-564, miR-154*, miR-7, miR-215, miR-382, miR-663, miR-516b, miR-99b, and miR-1276. In another specific embodiment, such kit comprises primers or probes specific for miR-374b*, miR-377*, miR-1285, and miR-1276. In yet another specific embodiment, such kit comprises primers or probes specific for miR-374b*, miR-377*, miR-1285, and miR-1204. In a further specific embodiment, such kit comprises primers or probes specific for miR-382, miR-1276, and miR-615-3p. In another specific embodiment, such kit comprises primers or probes specific for miR-215, miR-374b*, miR-382, miR-516b, and miR-7. In yet another specific embodiment, such kit comprises primers or probes specific for miR-382, miR-516b, and miR-7. Any of the above kits can optionally further comprise miRNA isolation or purification means. Any of the above kits can optionally further comprise instructions for use.

In a separate aspect, the invention provides a method for treatment of a melanoma recurrence (including distal metastasis and locoregional recurrence) in a subject in need thereof (e.g., human of experimental animal) comprising increasing the level and/or activity of at least one miRNA selected from the group consisting of miR-215, miR-374b*, miR-382, miR-516b, and miR-7 in the melanoma cells of the subject.

### BRIEF DESCRIPTION OF THE DRAWINGS

FIG. 1 illustrates a schematic depicting the flow of experiments in which the miRNAs are identified from the discovery cohort. The clinical parameters of the discovery cohort of primary melanomas are shown in Table 1. Information for the number of recurrent and non-recurrent patients is based on the following clinical criteria: stage at diagnosis, histologic subtype, thickness, ulceration, and follow up. Information is presented for all patients; only stage I or II patients with a minimum of 3 years of follow up are presented.

FIGS. 2A-H show the validation of array data by qRT-PCR. RNA samples of twenty tumors from the discovery cohort were used to validate the expression of 8 miRNAs by qRT-PCR. Resulting data was compared with the array expression data for the same samples and showed much of the array data (7 of 8 miRNAs tested) is robust and accurate. All data was centered on (referred to) an arbitrary sample (05-061). Relative expression of (A) hsa-let-7i (B) hsa-miR-16 (C) hsa-miR-21 (D) hsa-miR-29a (E) hsa-miR-146a (F) hsa-miR-146b-5p (G) hsa-miR-203 (H) hsa-miR-205 is plotted for both array and qRT-PCR data. R values were determined by Pearson correlation and are considered significant when r>0.444 with degrees of freedom of 18 and alpha<0.05.

FIGS. 3A-B show the relative cell invasion of the 40 candidate miRNAs (listed in Table 2) tested in the automated in vitro invasion screen in (A) 501MEL and (B) SK-MEL-147 cells. Invasion data is plotted as relative to a scrambled oligo control. miRNAs corresponding to white bars are the 5 invasion suppressive miRNAs. Data including the fold change and associated p values for each miRNA based on recurrent versus non-recurrent and thick versus thin tumors.

FIGS. 4A-C show a panel of melanoma cell lines in which ectopic expression of the indicated miRNA suppresses in vitro invasion. MicroRNA expression is decreased in recurrent versus non-recurrent tumors and/or thicker vs. thinner tumors. (A) Log2 expression ratios of indicated miRNA in recurrent versus non-recurrent tumors. Points indicate individual samples with mean and standard error of the mean (SEM) displayed. (B) Log2 expression ratios of indicated miRNA in tumors with progressively increasing thickness (<2 mm, 2 mm to 4 mm, >4 mm). Points indicate individual samples with mean and SEM displayed. (C) Quantification of in vitro invasion of the five indicated cell lines transfected with control or miRNA mimics. Results are represented as relative to SCR #1 scrambled oligo control. P values are determined by two-tailed t testing. *p=0.01 to 0.05, ** p=0.001 to 0.01, and *** p<0.001.

FIGS. 5A-E show the proliferation of cells ectopically expressing miRNA. Proliferation of SK-MEL-147, 501MEL, or SK-MEL-28 cells ectopically expressing (A) miR-215 (B) miR-374b* (C) miR-382 (D) miR-516b (E) miR-7 were analyzed. Relative cell proliferation is plotted normalized to a time zero control. P values are determined by two-tailed t testing at the specified time point. *p=0.01 to 0.05, ** p=0.001 to 0.01, and *** p<0.001.

FIGS. 6A-D show the suppression of in vivo lung metastasis in the presence of ectopic miRNA expression. Ectopically expressed miR-382 and miR-516b suppress in vivo lung metastasis of 451Lu xenografts. miR-516b also suppresses tumor growth in this model. (A) Tumor volume measurements of primary tumors plotted from initial measurement (day 14) to sacrifice (day 42) show miR-516b slows tumor growth. (B) Average tumor mass (mg) of primary tumors from control and treatment groups. P values are determined by two-tailed t testing. **p=0.001 to 0.01 (C) miR-382 and miR-516b suppress lung metastasis. Average number of macroscopic lung metastases quantified per field in 4 equivalent sized, randomly selected fields per animal. Lung metastases were assessed by GFP imaging. P values are determined by two-tailed t testing. **p=0.001 to 0.01, ***p<0.001 (D) Two representative macroscopic fluorescence images of mouse lungs per the indicated group show suppression of metastasis by miR-382 and miR-516b. Control is scrambled oligo.

FIGS. 7A-B show miRNA expression levels of lentiviral-transduced 451Lu cells relative to scrambled control for (A) miR-382, miR-516b, and miR-7. (B) Photographs of the primary tumors for scrambled control or the indicated miRNA-expressing 451Lu cells show that tumors grew to similar proportions. Lines represent one inch.

FIGS. 8A-D show macroscopic fluorescence images (inverted and duotone) of mouse lungs for each animal per group of the indicated scrambled control (A) miRH-SCR, or miRNA-expressing tumors: (B) miRH-382, (C) miRH-516b, and (D) miRH-7. Black spots are macroscopic lung metastases. Superimposed text indicates the weight (mg) of the corresponding primary tumor. Images are ordered from lightest to heaviest primary tumor.

FIGS. 9A-D show suppression of lung and liver metastasis of SK-MEL-147 cells by miR-7 implanted subcutaneously in NOG mice. (A) Tumor volume (cm³) measurements over time per the indicated miRNA groups. (B) Tumor mass (mg) at the time of sacrifice. (C) Average number of lung micrometastases per field per mouse. (D) Average number of macroscopic liver metastases as assessed by GFP imaging.

FIGS. 10A-D show the relative cell invasion of the 40 candidate siRNAs tested in the automated in vitro invasion screen in (A) 501MEL and (B) SK-MEL-128 (C) SK-MEL-147 or (D) 451Lu cells. Invasion data is plotted as relative to a scrambled oligo control. siRNAs shown as white bars are the 4 genes identified as direct targets of invasion suppressive miRNAs in FIG. 11. An siRNA directed to NEDD9 (gray) was used as a positive control.

FIGS. 11A-C show (A) suppression of in vitro invasion by siRNA-mediated depletion of direct miRNA targets, as assessed by (B) 3′UTR luciferase reporter assays. Quantification of in vitro invasion of the four indicated melanoma cell lines transfected with 50 nM control or (C) siRNA pool targeting the indicated gene. P values are determined by one-tailed t testing. *p=0.01 to 0.05, ** p=0.001 to 0.01, and *** p<0.001 Normalized relative light units from 3′UTR luciferase reporter assays. Predicted targeting miRNA (white bars) show clear 3′UTR repression. Significance is determined by 1-way ANOVA with Tukey's multiple comparison post-testing. *p=0.01 to 0.05, ** p=0.001 to 0.01, and *** p<0.001

FIGS. 12A-B indicate the putative miRNA targets that suppressed in vitro invasion, but were not confirmed as direct targets by 3′UTR luciferase reporter assays. (A) Relative cell invasion of SK-MEL-28, 501MEL, SK-MEL-147, and 451Lu cells after siRNA-mediated depletion of the indicated genes, MYO9B, AKT3, and RAC1. (B) 3′UTR luciferase reporter assay for the indicated gene.

FIGS. 13A-B show regulation of genes whose depletion suppressed invasion. NCAPG2, CTTN, and, CDC42 are unregulated during melanoma progression. Expression of the indicated gene from publically available data sets (Riker et al., BMC Medical Genomics, 2008, 1:13, and Talantov et al., Clinical Cancer Research, 2005, 11: 7234-7276) comparing (A) primary versus metastatic melanoma or (B) nevi vs melanoma. P values are determined by two-tailed, Mann-Whitney t testing. *p=0.01 to 0.05, ** p=0.001 to 0.01, and *** p<0.001.

FIGS. 14A-D indicate a 21 miRNA, tissue-based expression signature predicting recurrence of stage I and II patients at the time of diagnosis. (A) Receiver operating characteristic (ROC) curves of the discovery and validation cohorts for the discrimination of recurrent vs non-recurrent stage I and II tumors using clinical variables (stage, thickness, and ulceration). (B) ROC curves of the discovery and validation cohorts for the discrimination of recurrent vs non-recurrent stage I and II tumors using the described 21 miRNA expression signature. AUC=97% and 95%, respectively. (C) Kaplan-Meier curves of recurrence-free survival for low and high risk groups determined from ROC curves. The number of events is indicated between brackets. Significance was determined by log-rank test. p<0.001 (D) Waterfall plots of recurrent and non-recurrent patients sorted by risk score determined from predictive miRNA expression signature. The clinical parameters of the validation cohort of primary melanomas are shown in Table 3. Information for the number of recurrent and non-recurrent patients is based on the following clinical criteria: stage at diagnosis, histologic subtype, thickness, ulceration, and follow up. Information is presented for all patients and only for patients with stage I or II having a minimum of 3 years of follow up.

FIGS. 15A-C show a correlation between miRNA expression and overall survival. Expression of miRNAs hsa-miR-374b* and hsa-miR-516b correlates with overall survival (melanoma-specific death as endpoint) in a thickness-adjusted, multivariate Cox Proportional Hazards model. (A) Table of hazard ratios and p values associated with overall survival in discovery and validation cohorts, and the combined p-value by the Fisher's method. Kaplan-Meier survival curves of patients whose primary tumors expressed (B) hsa-miR-374b* or (C) hsa-miR-516b above (high) or below (low) the median. P values are determined by Log-rank test.

FIGS. 16A-B show inhibition of miR-382 and miR-516b enhances invasion of poorly invasive melanoma cells. (A) Relative cell invasion of WM35 cells transfected with the indicated control or miRNA inhibitor(s) (B) Relative luciferase activity of the indicated miRNA sensors co-transfected with miRNA mimic in the presence of control or miRNA inhibitor.

FIGS. 17A-D show the area under the receiver operating characteristic (ROC) curve of the discovery cohort and the validation cohort deduced from four logistic regression models. Each of the four models achieved an area under the ROC between 94% and 96% in the discovery cohort and between 84% and 96% in the validation cohort.

### DETAILED DESCRIPTION OF THE INVENTION

The current invention is based on the observation that there are different levels of certain miRNAs in different patients at the time of diagnosis of melanoma. As discussed in more detail in the Examples, below, 204 primary melanoma tumors were analyzed, and top differentially-expressed miRNAs in recurrent versus non-recurrent patients were identified, as well as miRNAs whose expression correlated with tumor thickness (Tables 4a-d). Selected miRNAs were further tested in an in vitro screen in two melanoma cell lines to determine which miRNAs functionally impact melanoma metastasis. A subsequent screen of 13 of the miRNAs in both in vitro invasion and cell proliferation assays revealed miR-215, miR-374b*, miR-382, miR-516b, and miR-7 as being less expressed in recurrent as opposed to non-recurrent and/or thicker versus thinner tumors and were deemed potent suppressors of in vitro invasion. It has been shown that alteration of miRNA expression correlates with cancer progression, and the perturbation of individual miRNAs can functionally impact cancer cell metastasis.

Based on these observations, the present invention provides a novel highly sensitive method for predicting the likelihood of recurrence of melanoma (including distal metastasis and locoregional recurrence) at the time of diagnosis in a subject, said method comprising:

a. measuring the levels of four or more miRNAs selected from the group consisting of miR-10a, miR-1285, miR-377*, miR-513b, miR-342-3p, miR-625*, SNORD3A, miR-1204, miR-574-3p, let-7a-2*, miR-615-3p, miR-564, miR-154*, miR-663, miR-99b, miR-1276, miR-215, miR-374b*, miR-382, miR-516b, and miR-7, in a melanoma sample collected from the subject;

b. calculating combined levels of the miRNAs measured in step (a);

c. comparing the combined levels of the miRNAs measured in step (a) with the corresponding combined control levels of said miRNAs, and

d. (i) identifying the subject as being at high risk of melanoma recurrence if the combined levels of the miRNAs measured in step (a) are higher than the corresponding combined control levels or (ii) identifying the subject as being at low risk of melanoma recurrence if the combined levels of the miRNAs measured in step (a) are same or lower than the corresponding combined control levels.

The method of the invention makes possible early prognosis of recurrence of melanoma, e.g., at the time of the diagnosis prior to occurrence of major morphological changes and/or metastasis associated with the disease, allowing for early application of treatments to prevent such morphological changes and/or metastasis. Patients determined to be at low risk of melanoma recurrence will likely receive no additional treatment after primary tumor excision, and will simply be subject to annual or semi-annual check-ups (e.g., physical and skin screening). On the other hand, patients determined to be at high risk of melanoma recurrence, after primary tumor excision, will be likely subject to a more frequent and detailed surveillance (e.g., involving MRI or other imaging modality to identify local and distal recurrences), more extensive primary tumor staging (e.g., involving sentinel and/or regional lymph node mapping) and may be subject to a melanoma treatment (e.g., Interleukin 2 (IL2), Aldesleukin (Proleukin), Dacarbazine (DTIC-Dome), Ipilimumab (Yervoy), temozolomide, Vemurafenib (Zelboraf), and any combinations thereof).

The method of the invention also allows for more precise identification of various groups of patients who can be then recruited in clinical trials to develop and/or test new treatments to prevent melanoma recurrence.

In addition, cellular pathways regulated by the prognostic miRNAs identified herein are potential molecular therapeutic targets for control of melanoma recurrence. Thus, in conjunction with the prognostic method, the present invention also provides a method for treatment of a melanoma recurrence in a subject in need thereof comprising increasing the level and/or activity of at least one miRNA selected from the group consisting of miR-215, miR-374b*, miR-382, miR-516b, and miR-7 in the melanoma cells of the subject. Such increase in the level and/or activity of said miRNAs can be achieved using any method known in the art (e.g., over-expressing miRNA or mature miRNA mimic [an oligonucleotide, usually with some structural change(s), of the same sequence as the mature miRNA], e.g., using viral constructs; inhibiting negative or activating positive miRNA regulators [transcriptional or epigenetic], etc.).

The methods of the invention involve measuring miRNA levels. Examples of useful methods for measuring miRNA level in solid tumors include hybridization with selective probes (e.g., using Northern blotting, bead-based flow-cytometry, oligonucleotide microchip [microarray] (e.g., from Agilent, Exiqon, Affymetrix), or solution hybridization assays such as Ambion mirVana miRNA Detection Kit), polymerase chain reaction (PCR)-based detection (e.g., stem-loop reverse transcription-polymerase chain reaction [RT-PCR], quantitative RT-PCR based array method [qPCR-array]), or direct sequencing by one of the next generation sequencing technologies (e.g., Helicos small RNA sequencing, miRNA BeadArray (Illumina), Roche 454 (FLX-Titanium), and ABI SOLiD). For review of additional applicable techniques see, e.g., Chen et al., BMC Genomics, 2009, 10:407; Kong et al., J Cell Physiol. 2009; 218:22-25.

In some embodiments, miRNAs are purified prior to quantification. miRNAs can be isolated and purified from solid tumors by various methods, including, e.g., Qiazol or Trizol extraction or the use of commercial kits (e.g., miRNeasy kit [Qiagen], MirVana RNA isolation kit [Ambion/ABI], miRACLE [Agilent], High Pure miRNA isolation kit [Roche], and miRNA Purification kit [Norgen Biotek Corp.]), concentration and purification on anion-exchangers, magnetic beads covered by RNA-binding substances, or adsorption of certain miRNA on complementary oligonucleotides.

In some embodiments, miRNA degradation in solid tumor samples and/or during miRNA purification is reduced or eliminated. Useful methods for reducing or eliminating miRNA degradation include, without limitation, adding RNase inhibitors (e.g., RNasin Plus [Promega], SUPERase-In [ABI], etc.), use of guanidine chloride, guanidine isothiocyanate, N-lauroylsarcosine, sodium dodecylsulphate (SDS), or a combination thereof. Reducing miRNA degradation in samples is particularly important when sample storage and transportation is required prior to miRNA quantification.

In conjunction with the prognostic method, the present invention also provides various kits comprising primer and/or probe sets specific for the detection of biomarker miRNAs. Non-limiting examples of primer or probe combinations in kits are as follows:

1. Primers or probes specific for four or more miRNAs selected from the group consisting of miR-10a, miR-1285, miR-374b*, miR-377*, miR-513b, miR-342-3p, miR-625*, SNORD3A, miR-1204, miR-574-3p, let-7a-2*, miR-615-3p, miR-564, miR-154*, miR-7, miR-215, miR-382, miR-663, miR-516b, miR-99b, and miR-1276.
2. Primers or probes specific for miR-10a, miR-1285, miR-374b*, miR-377*, miR-513b, miR-342-3p, miR-625*, miR-1204, miR-574-3p, let-7a-2*, miR-615-3p, miR-564, miR-154*, miR-7, miR-215, miR-382, miR-663, miR-516b, miR-99b, and miR-1276.
3. Primers or probes specific for miR-374b*, miR-377*, miR-1285, and miR-1276.
4. Primers or probes specific for miR-374b*, miR-377*, miR-1285, and miR-1204.
5. Primers or probes specific for miR-382, miR-1276, and miR-615-3p.
6. Primers or probes specific for miR-215, miR-374b*, miR-382, miR-516b, and miR-7.
7. Primers or probes specific for miR-382, miR-516b, and miR-7.

Such kits can be useful for direct miRNA detection in primary melanoma tumor samples isolated from patients or can be used on purified miRNA samples.

A kit of the invention can also provide reagents for primer extension and amplification reactions. For example, in some embodiments, the kit may further include one or more of the following components: a reverse transcriptase enzyme, a DNA polymerase enzyme (such as, e.g., a thermostable DNA polymerase), a polymerase chain reaction buffer, a reverse transcription buffer, and deoxynucleoside triphosphates (dNTPs). Alternatively (or in addition), a kit can include reagents for performing a hybridization assay. The detecting agents can include nucleotide analogs and/or a labeling moiety, e.g., directly detectable moiety such as a fluorophore (fluorochrome) or a radioactive isotope, or indirectly detectable moiety, such as a member of a binding pair, such as biotin, or an enzyme capable of catalyzing a non-soluble colorimetric or luminometric reaction. In addition, the kit may further include at least one container containing reagents for detection of electrophoresed nucleic acids. Such reagents include those which directly detect nucleic acids, such as fluorescent intercalating agent or silver staining reagents, or those reagents directed at detecting labeled nucleic acids, such as, but not limited to, ECL reagents. A kit can further include miRNA isolation or purification means as well as positive and negative controls. A kit can also include a notice associated therewith in a form prescribed by a governmental agency regulating the manufacture, use or sale of diagnostic kits. Detailed instructions for use, storage and troubleshooting may also be provided with the kit. A kit can also be optionally provided in a suitable housing that is preferably useful for robotic handling in a high throughput setting.

The components of the kit may be provided as dried powder(s). When reagents and/or components are provided as a dry powder, the powder can be reconstituted by the addition of a suitable solvent. It is envisioned that the solvent may also be provided in another container. The container will generally include at least one vial, test tube, flask, bottle, syringe, and/or other container means, into which the solvent is placed, optionally aliquoted. The kits may also comprise a second container means for containing a sterile, pharmaceutically acceptable buffer and/or other solvent.

Where there is more than one component in the kit, the kit also will generally contain a second, third, or other additional container into which the additional components may be separately placed. However, various combinations of components may be comprised in a container.

Such kits may also include components that preserve or maintain DNA or RNA, such as reagents that protect against nucleic acid degradation. Such components may be nuclease or RNase-free or protect against RNases, for example. Any of the compositions or reagents described herein may be components in a kit.

### DEFINITIONS

As used herein in connection with melanoma, the term “recurrence” refers to a return of the disease, either locally (e.g., where it used to be before resection) or distally (e.g., metastasis).

The terms “microRNA” or “miRNA” as used herein refer to a class of small approximately 22 nt long non-coding RNA molecules. They play important roles in the regulation of target genes by binding to complementary regions of messenger transcripts (mRNA) to repress their translation or regulate degradation (Griffiths-Jones Nucleic Acids Research, 2006, 34, Database issue: D140-D144). Frequently, one miRNA can target multiple mRNAs and one mRNA can be regulated by multiple miRNAs targeting different regions of the 3′ UTR. Once bound to an mRNA, miRNA can modulate gene expression and protein production by affecting, e.g., mRNA translation and stability (Baek et al., Nature 455(7209):64 (2008); Selbach et al., Nature 455(7209):58 (2008); Ambros, 2004, Nature, 431, 350-355; Bartel, 2004, Cell, 116, 281-297; Cullen, 2004, Virus Research., 102, 3-9; He et al., 2004, Nat. Rev. Genet., 5, 522-531; and Ying et al., 2004, Gene, 342, 25-28). Information on most currently known miRNAs can be found in the miRNA database miRBase (available at the world wide web at mirbase.org). For the purposes of the present invention, the terms “microRNA” or “miRNA” include, in addition to the miRNAs described above, SNORD3A small RNA.

The term “miRNA array” refers to a multiplex technology used in molecular biology and in medicine. It consists of an arrayed series of multiple (e.g., up to 2000) microscopic spots of oligonucleotides, each containing a specific sequence (probe) complementary to a particular target miRNA. After probe-target hybridization under high-stringency conditions the resulting hybrids are usually detected and quantified by quantifying fluorophore-, silver-, or chemiluminescence-labeled targets to determine relative abundance of miRNA. In the methods of the present invention, both custom-made and commercially available miRNA arrays can be used. Non-limiting examples of useful commercially available miRNA arrays (based on various methods of target labeling, hybrid detection and analysis) include arrays produced by Exiqon, Affymetrix, Agilent, Illumina, Invitrogen, Febit, and LC Sciences.

The term “next generation sequencing technologies” broadly refers to sequencing methods which generate multiple sequencing reactions in parallel. This allows vastly increased throughput and yield of data. Non-limiting examples of commonly used next generation sequencing platforms include Helicos small RNA sequencing, miRNA BeadArray (Illumina), Roche 454 (FLX-Titanium), and ABI SOLiD.

The term “combined levels of the miRNAs” as used herein refers to the linear combinations of miRNAs levels. The linear combinations of miRNAs levels can be calculated using any method known in the art. For example, as specified in the Examples section, below, the coefficients required for such linear combinations can be calculated using the logistic regression method, i.e., β1*x1+β2*x2+ . . . +βk*xk, where β's are the coefficients from logistic regression model, and x's are the levels of miRNAs (see, e.g., Steyerberg (2009) Clinical Prediction Models, Springer, N.Y.). The coefficients can be positive or negative. The “combined levels”, or the score, is always positively associated with the risk of recurrence. Thus, patients with higher score have a higher probability of recurrence. The logistic regression method can also include more clinical predictors if necessary. The performance of logistic regression models is measured using the area under the Receiving Operative Characteristic (ROC) curves: the larger the area under the ROC curve, the better performance of the model. The best performed model would yield the coefficients required to calculate the linear combination of levels of candidate miRNAs.

In the context of the present invention insofar as it relates to melanoma and melanoma recurrence, the terms “treat”, “treatment”, and the like mean to relieve or alleviate at least one symptom associated with such condition, or to slow or reverse the progression of melanoma or melanoma recurrence, or to arrest, prevent or delay the onset (i.e., the period prior to clinical manifestation) and/or reduce the risk of developing or worsening of melanoma or melanoma recurrence.

An “individual” or “subject” or “animal”, as used herein, refers to humans, veterinary animals (e.g., cats, dogs, cows, horses, sheep, pigs, etc.) and experimental animal models of melanoma. In a preferred embodiment, the subject is a human.

The term “purified” as used herein refers to material that has been isolated under conditions that reduce or eliminate the presence of unrelated materials, i.e., contaminants, including native materials from which the material is obtained. For example, RNA purification includes elimination of proteins, lipids, salts and other unrelated compounds. Besides, for some methods of analysis a purified miRNA is preferably substantially free of other RNA oligonucleotides contained in tumor samples (e.g., rRNA and mRNA fragments, etc.). As used herein, the term “substantially free” is used operationally, in the context of analytical testing of the material. Preferably, purified material substantially free of contaminants is at least 50% pure; more preferably, at least 90% pure, and still more preferably at least 99% pure. Purity can be evaluated by chromatography, gel electrophoresis, composition analysis, biological assay, and other methods known in the art.

As used herein, the term “similarly processed” refers to samples (e.g., tumor samples or purified miRNAs) which have been obtained using the same protocol.

The term “associated with” is used to encompass any correlation, co-occurrence and any cause-and-effect relationship.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

In accordance with the present invention there may be employed conventional molecular biology, microbiology, and recombinant DNA techniques within the skill of the art. Such techniques are explained fully in the literature (e.g., ref 32-59).

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

### Materials and Methods

### Cell Culture

501MEL cells (Halaban et al., PLoS One. 2009, 4(2):e4563) were obtained from Yale University and were cultured in Optimem+5% fetal bovine serum (FBS). 451Lu cells derived from metastatic melanoma were obtained from Dr. Meenhard Herlyn at Wistar Institute (Smalley et al., Mol. Cancer Ther., 2006, 5(5):1136-1144). The cells were cultured in Tu2%, which contains 80% MCDB153 (Sigma Aldrich) and 20% L15 (Cellgro), and were supplemented with ˜2% FBS, 1.68 mM CaCl₂, and 5 μg/mL bovine insulin. SK-MEL-147, SK-MEL-173, and SK-MEL-28 cells were obtained from Dr. Alan Houghton at Memorial Sloan-Kettering Cancer Center (see Houghton et al., J Exp Med., 1982, 156(6):1755-1766 and Segura et al., Proc. Natl. Acad. Sci. USA, 2009, 106(6):1814-1819) and were cultured in DMEM+10% FBS. All cells were grown in a humidified incubator at 37° C. and 5% CO₂.

### RNA Extraction

Pelleted cells were stored at −20° C. until RNA extraction. RNA was extracted using miRNeasy mini kits (Qiagen) following manufacturer's recommendations. Briefly, pelleted cells were thawed on ice. 700 μL per tube of Qiazol (Qiagen) was added, tubes were vortexed for ˜60 sec, and incubated at room temperature for 5 minutes. 140 μL chloroform was added and tubes were shaken for 15 sec, followed by 2 minutes incubation at room temperature. Tubes were centrifuged at 12,000×g at 4° C. for 15 minutes. Aqueous phase (˜350 uL) was transferred to a fresh microcentrifuge tube. 1.5× volumes of 100% EtOH were added and mixed by vortexing briefly. 700 μL at a time were transferred to RNeasy mini spin columns and centrifuged at 13,000 rpm for 30 sec. Repeat with remainder of sample, discarding flow-through. 350 μL of buffer RWT were added per column and centrifuged at 13,000 rpm for 30 seconds. Flow-through was discarded. 80 μL of DNAse I (Qiagen) was added to each column and incubated at room temperature for 15 minutes. 350 μL of buffer RWT were added to column, followed by centrifugation at 13,000 rpm for 30 sec. Two washes with 500 μL buffer RPE were performed discarding flow-through each time, followed by centrifugation at 13,000 rpm for 2 minutes to remove all traces of ethanol from RPE buffer. Columns were transferred to 1.5 mL RNA collection tubes and 30 to 50 μL RNase-free H₂O was added per column for RNA elution. After 1 minute incubation at room temperature, columns were centrifuged at 13,000 rpm for 1 minute. Eluted RNA was quantified by Nanodrop 2000 (Thermo Scientific) and stored at −80° C.

FFPE Melanomas or Nevi.

5 μm sections (4-12) were attached to PEN-Membrane 2.0 μm slides (Leica) designed for laser capture microdissection. Primary melanoma tissues were macroscopically dissected using disposable scalpels (Feather No. 11) under a dissecting microscope and guided by hematoxylin and eosin (H&E) staining of consecutive sections. Cut sections were stored in microcentrifuge tubes until RNA extraction. RNA extraction was performed with miRNeasy FFPE kit (Qiagen) following manufacturer's recommendations. RNA was quantified by Nanodrop 2000 (Thermo Scientific) and stored at −80° C.

miRNA Array Profiling

miRNA expression profiling of FFPE-extracted RNA from primary melanomas was performed by Exiqon. Briefly, the quality of the total RNA was verified by an Agilent 2100 Bioanalyzer profile (Agilent). For each cohort, a reference sample was generated by mixing an equal amount of all samples analyzed. 300 ng total RNA from sample and reference was labeled with Hy3™ and Hy5™ fluorescent label, respectively, using the miRCURY™ LNA Array power labelling kit (discovery cohort) or miRCURY LNA™ microRNA Hi-Power Labeling Kit (validation cohort) (Exiqon, Denmark) by following the procedure described by the manufacturer. The Hy3™-labeled samples and a Hy5™-labeled reference RNA sample were mixed pair-wise and hybridized to the miRCURY™ LNA array version 11.0 (discovery cohort) or miRCURY LNA™ microRNA Array 6th generation (validation cohort) (Exiqon, Denmark), which contain capture probes targeting all miRNAs for human, mouse or rat registered in the miRBASE version 14.0 or 16.0, respectively, at the Sanger Institute (http://www.mirbase.org/). The hybridization was performed according to the miRCURY™ LNA array manual using a Tecan HS4800 hybridization station (Tecan, Austria). After hybridization, the microarray slides were scanned and stored in an ozone-free environment (ozone level below 2.0 ppb) in order to prevent potential bleaching of the fluorescent dyes. The miRCURY™ LNA array microarray slides were scanned using the Agilent G2565BA Microarray Scanner System (Agilent Technologies, Inc., USA) and the image analysis was carried out using the ImaGene 8.0 or 9.0 software (BioDiscovery, Inc., USA). The quantified signals were background corrected (Normexp with offset value 10—Ritchie et al., Bioinformatics, 2007, 23(20):2700-2707) and normalized using the global Lowess (LOcally WEighted Scatterplot Smoothing) regression algorithm (Cleveland, W. S., 1979, J. Amer. Statist. Assoc., 74:829-836).

### Statistical Analyses

After Lowess normalization, scale normalization was performed such that each array has the same median expression level and inter-quartile range 1 (the third quartile minus the first quartile). The 339 miRNAs with highest expression levels (based on both Hy3 and Hy5 signals) among 867 miRNA probes in the discovery cohort were used for variable selection.

Predictive Signature for Recurrence from Discovery Cohort.

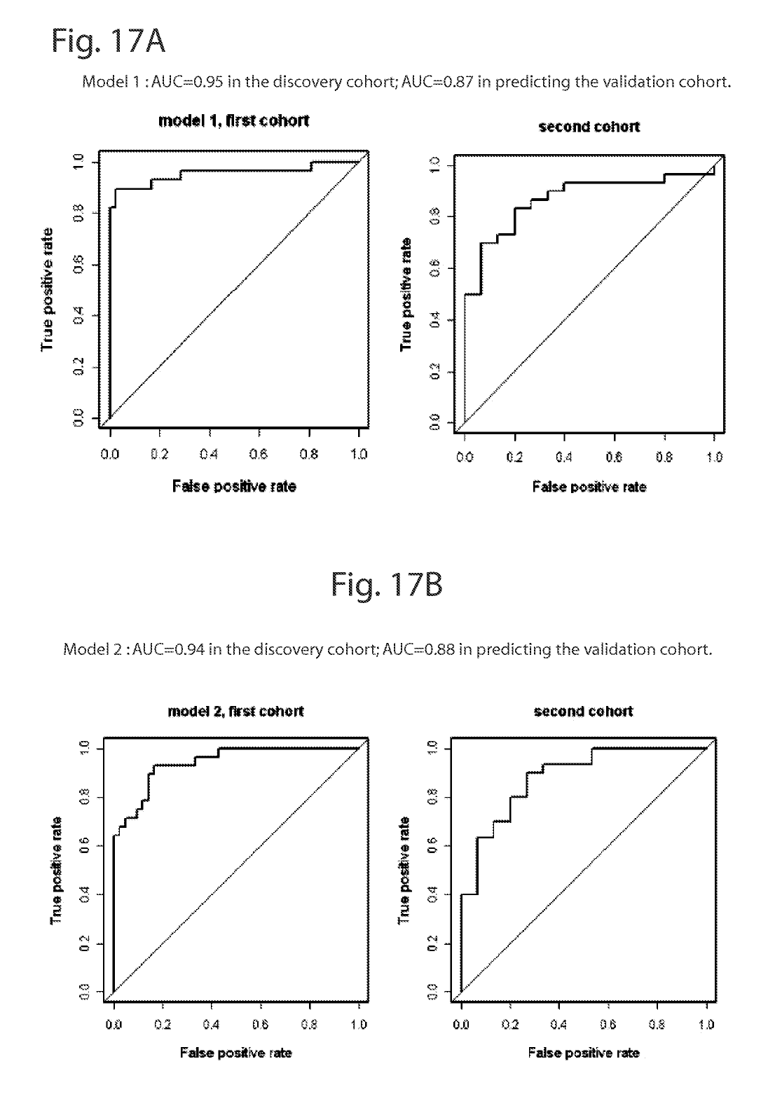

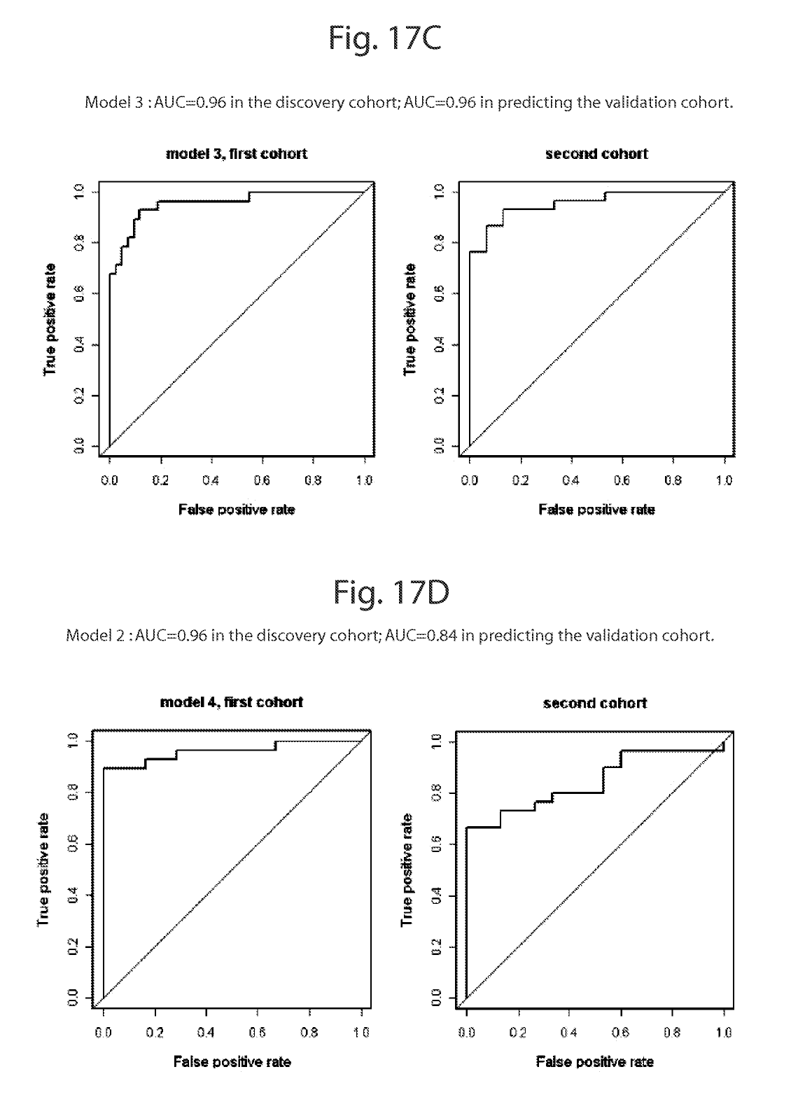

Seventy stage I/II patients with at least 3 years of follow-up in the discovery cohort were used to identify a predictive signature for 3-year recurrence. The miRNAs were ranked (adjusted for stage) according to three related endpoints: 3-year recurrence (logistic regression, Table 4a), tumor thickness (linear regression, Table 4b), and recurrence-free survival (RFS) (Cox PH regression, Table 4c). Starting from top ranked miRNAs in each of the rank list, multiple logistic regression models were developed using miRNAs as predictors with adjustment for stage. Since the cohort size is relatively small, each significant model has 7 to 11 miRNAs constructed by maximizing the area under the receiver operating characteristic (ROC) curve for 3-year recurrence with 4-fold cross validation. To minimize premature exclusion of promising miRNAs, 4 logistic regression models (FIG. 17 and Table 4 a-d) were selected with a combined total of 21 miRNAs, as the predictive signature set. The linear combination of predictors in each logistic model was used to provide the risk score. The risk scores from the 4 selected regression models were averaged to give an integrated risk score (classifier) which can be used to classify patients into high and low risk groups and form an ROC curve. Using maximum Youden's index (i.e., sensitivity+specificity−1) of the ROC as a cut-off point, Kaplan-Meier RFS curves for the resulting high and low risk groups were plotted, and log-rank test was used to compare the two curves.

Evaluation of the Predictive Signature in the Validation Cohort.

For independent validation, the recurrence potential score formula obtained from the discovery cohort were directly applied to the validation cohort and used to obtain an ROC, the same cut-off point was used to classify the validation cohort patients into high and low risk groups. Kaplan-Meier curves were plotted for the RFS of the two groups and log-rank test p-value was obtained.

From the discovery cohort, differential expression of miRNAs between thick and thin or recurrent and non-recurrent tumors was determined using two-tailed t-testing using the Benjamini Hochberg method (Benjamini, Yoav and Hochberg, Yosef, Journal of the Royal Statistical Society, Series B (Methodological), 1995, 57(1): 289-300).

### Real Time PCR (RT-PCR)

Validation of miRNA expression in tissues.

Mature miRNA expression validation in RNA extracted from FFPE primary melanomas was performed using Exiqon reagents following manufacturer's recommendations. 20 samples from discovery cohort were used for analysis. Reverse transcription was performed using miRCURY LNA Universal cDNA synthesis kit (Exiqon). Briefly, 25 ng RNA was diluted to 10 μL in nuclease-free H₂O in 96-well PCR plates (Biorad). Reverse transcription master mixes were made with 5× reaction buffer, H₂O, Spike RNA control, and +/− reverse transcriptase. 10 μL master mix was added to each 10 μL aliquot of diluted RNA and mixed gently. Tubes were incubated for 60 minutes at 42° C. followed by 5 minutes at 95° C. Duplicate room temperature reactions were performed for each sample tested. Reverse transcription products were used immediately or briefly stored at −20° C. until use. PCR was carried out using miRCURY LNA SYBR green mastermix and miRNA-specific LNA primers. Briefly, cDNA was diluted 80× in nuclease-free H₂O and ROX passive reference dye (Invitrogen). PCR master mixes were made with 2× miRCURY LNA SYBR green master mix (5 μL) and LNA primers (1 μL). 4 μL diluted cDNA or H₂O was added to wells of 384-well plates containing 6 μL of PCR master mix. Duplicate wells of each cDNA were run. PCR reactions were performed using a 7900 HT (Applied Biosystems) as follows: 10 min at 95° C., and 40 cycles of 10 sec at 95° C. followed by 60 sec at 60° C. with ramp rate of 1.6° C./sec. Data was analyzed in Excel (Microsoft) and relative expression determined by the method of Livak (Livak K J, Schmittgen T D, Methods. 2001, 25(4):402-408). Ct values from duplicate room temperature reactions were averaged. The data were normalized to the geometric mean of 3 internal reference miRNAs (miR-146b-3p, miR-let-7e, and miR-485-3p) selected due to their low deviation across samples in the original arrays and to the RNA spike-in control (as a measure of room temperature efficiency). Array log 2 expression ratios and qPCR expression was expressed as relative to an arbitrary sample (05-061) and plotted using Graphpad PRISM (Graphpad; www.graphpad.com). Correlation (R) values were calculated by Pearson correlation.

miRNA Overexpression in Cultured Cells

Mature miRNA expression was quantified using Taqman miRNA assays (Applied Biosystems) following manufacturer's recommendations. Briefly, RNA was diluted to 12.5 ng/μL. miRNA-specific reverse transcription (room temperature) master mixes were made with H₂O, 10× RT buffer, miRNA RT primers, RNase inhibitor, dNTPs, and with or without reverse transcriptase. 14 μL RT master mix was added per well and 1 μL appropriate 12.5 ng/μL RNA stock was added to master mix, followed by incubation on ice for 5 minutes. Reverse transcription (RT) was carried out in a thermal cycler with 30 min at 16° C., 30 minutes at 42° C., and 15 minutes at 85° C. RT products were stored at −20° C. if not used immediately. Polymerase chain reaction (PCR) master mixes were made with miRNA-specific 20× Taqman primers, (2×) Taqman universal PCR master mix, fluorescein (Molecular Probes), and H₂O. 18.66 μL PCR master mix was added per well in 96-well PCR plates (Biorad) followed by addition of 1.33 μL per well of appropriate RT or −RT reaction product or H₂O. PCR reaction was performed on an iCycler equipped with a MyIQ Real-time PCR Imaging system (Biorad). Cycling was performed as follows: 10 sec and 95° C., and 40 cycles of 15 sec at 95° C. and 60 sec at 60° C. Ct threshold was selected with amplification curves in log scale. Relative expression were analyzed by Livak method using U6 snRNA or RNU44 as internal controls and plotted with Graphpad PRISM (Graphpad; www.graphpad.com).

### Viral Production

4×10⁶ 293 T cells were seeded per 10 cM tissue culture dish and incubated overnight at 37° C. and 5% CO₂. 16-20 hrs after seeding, 293T were co-transfected with lentiviral expression constructs (15 μg), viral packaging plasmid (psPAX2, 10 μg), and viral envelope plasmid (pMD2.G, 5 μg) using Lipofectamine2000 (Invitrogen) following manufacturer's recommendations. Viral supernatant was collected and 0.45 μm filtered at 36 hrs post-transfection and stored at 4° C. for short-term use (1-5 days) or −20° C. for long-term storage (5-30 days).

### Viral Transduction

Target cells were seeded and incubated overnight prior to infection. Medium was replaced with 1:2 diluted viral supernatant with 4 μg/mL polybrene and incubated for 6-8 hrs, followed by replacement with growth medium. Cells were checked for GFP expression on subsequent days to ensure pure populations of GFP-bright transduced cells.

### Invasion Assay Screen

Fluorescent Cell Generation.

Lentiviral supernatant was generated as previously described (Segura et al., Proc. Natl. Acad. Sci. USA, 2009, 106(6):1814-1819) of green fluorescent protein (GFP) expression constructs (pGIPZ, Openbiosystems or pMIRH, Systems Biosciences). All cell lines were transduced at high efficiency to generate pure, GFP bright cell populations for use in invasion assays.

Reverse Transfection.

Transfection conditions were optimized for each cell line using dy547 or fluorescein labeled oligos (Dharmacon, dy547). Liposomal transfection complexes with miRNA mimics (Dharmacon, 50 nM final) or siRNA pools (Dharmacon, Smart Pools, 50 nM) were generated with Lipofectamine 2000 (Invitrogen, 0.2 μL per well) in at least triplicate in 96-well plates following manufacturer's recommendations. Replicate wells were scattered on the plate to limit technical bias. GFP expressing cells were seeded at specific densities (501MEL-25,000 cells/well, SK-MEL-147, SK-MEL-28, and 451Lu—30,000 cells/well, SK-MEL-173-40,000 cells/well) into wells containing liposomal complexes followed by overnight incubation in a humidified incubator at 37° C. and 5% CO₂. Media was changed after incubation with liposomal complexes. 48-hours after initiation of transfection, cells were used for invasion assay seeding.

Invasion Assay Seeding.

Optimization was performed for each cell line to identify assay time length. Additionally, to identify the optimal seeding density, a 2-fold dilution series of each cell line was performed to test the linear range of the assay. 48 hour post-transfection cell counts were performed in initial experiments to ensure optimal cell quantities were transferred from transfection plate to invasion assay plate. Prior to invasion assay seeding, 96-well Fluoroblok inserts (Becton Dickinson) were coated with 10 μg/mL fibronectin in PBS for 60 min at room temperature, followed by PBS supplemented with 2.5% bovine serum albumin at RT until cell seeding (10-30 minutes). Cells were washed 1× with PBS, dissociated from 96-well plates using small volumes of 0.05% Trypsin-EDTA (Invitrogen) or Cell Dissociation Buffer (PBS-based, Invitrogen), and quenched with the described basal growth media, but supplemented with only 1/10 the volume of FBS and bovine insulin (451Lu) (top chamber media). Single cell suspensions were generated by gentle, repetitive (40×) pipetting using an 8-channel multipipette. 12.5 μL, 25 μL, or 50 μL of cell suspension (cell line dependent) were transferred using an 8-channel multipipette to the upper chamber of the 96-well Fluorblok inserts to yield resulting cell inputs in the previously defined optimal range. Top chamber media was supplemented to 50 μL for each insert well. Cells were allowed to settle then 200 μL growth media per well was added to the lower chamber of 96-well Fluoroblok inserts. An equivalent volume of cell suspension as used in the invasion assay was transferred to a standard 96-well tissue culture microplate as a cell input control. Invasion assay and cell input control plates were maintained at 37° C. and 5% CO₂ until automated assay quantification. Cell input control plates were imaged and counted ˜30 min after seeding, except SK-MEL-173 which was imaged and counted at 40 hrs post-seeding. Invasion assay plates were imaged and counted 8-20 hrs post-seeding, except SK-MEL-173, which was imaged and counted at 40 hrs post-seeding.

Invasion Quantification.

Invasion assay and cell input control plates were scanned using a Cellomics ArrayScan VTI HCS Reader (Cellomics), a high-content inverted fluorescent microscope system with companion software. A 5× objective was used for imaging. Four fields per insert, which covered >95% of the insert membrane bottom were imaged for invasion assay plates, while seven fields per well were imaged for cell input control plates. GFP-labeled cells were counted by GFP fluorescence using a version of the TargetActivation_v3 protocol (Cellomics) modified to optimally capture individual cells. Modified parameters included: fixed threshold of 25-50, exposure length, size exclusion criteria, smoothing factor, and segmentation. Cell counts for each well were normalized to the average counts (of replicate wells) for the corresponding condition in the cell input plate to control cell proliferation effects that may have occurred between initiation of transfection and assay seeding.

Cell Proliferation.

Indicated cells were reverse transfected (n=6) following previously established conditions. 48-hours after transfection, cells were washed 1× with PBS, dissociated from well with 30 μL per well of 0.05% Trypsin-EDTA (Invitrogen). Trypsin was quenched with 270 μL growth media. 30-50 μL per well were transferred to replicate plates (n=6) to initiate growth curve. After cell attachment (4-6 hrs), 1 plate was fixed as a zero time point, and subsequent plates were fixed every 24 hours thereafter. Plates were fixed with 1% glutaradlehyde (Sigma) in PBS for 15 minutes at room temperature, followed by storage in PBS at 4° C. All plates were stained with 0.5% crystal violet in PBS for 2 hrs followed by extensive wash with diH₂O. Crystal violet staining was dissolved in 15% acetic acid and measured at absorbance 595 nm in a standard plate reader. Data are plotted as relative proliferation normalized to time zero for each condition.

In Vivo Experiments.

451Lu cells transduced with lentiviral supernatants containing miRNA expression constructs (miRH backbone, Systems Biosciences) were resuspended in growth media at a concentration of 2×10⁶ cells/150 μL, aliquoted into eppendorf tubes (150 μL) and maintained on ice until injection. Immediately prior to injection, cell aliquots were mixed with 150 μL Matrigel (Becton Dickinson). Cell/Matrigel suspensions were injected subcutaneously in the right flank of NOD/Shi-scid/IL-2Rγnull (NOG) mice (Jackson Laboratory) (n=9 per group). When tumors were palpable (14 days), length and width measurements were made with calipers 3 times weekly until the animals were sacrificed. Tumor volume was calculated by the following formula: a²*b/2, where a is the width and b is the length. Tumor did not develop in one animal of the miR-374b/b* group for technical reasons and was discarded from subsequent analyses. 6 weeks after cell injection all animals were sacrificed to assess tumor mass and quantify lung metastasis. Tumors were extracted, weighed and imaged. Lungs, liver, spleen, and kidney were removed for analysis of metastasis. Ventral and dorsal macroscopic images of metastasis-bearing lungs were taken with a fluorescent dissecting microscope equipped with a black and white camera. Images were processed in Photoshop (Adobe) by inversion followed by conversion to duotone. Duotone parameters for black were adjusted as follows: 5:10%, 10:30%, 20:70%, 30:100%. Macroscopic metastases were quantified by counting lesions in 4 boxes of equal size (210 by 210 pixels) per lung per side and averaged per mouse. Data were plotted using Graphpad PRISM and significance determined by one-tailed t-testing.

3′UTR Reporter Luciferase Assay.

Full length 3′UTR luciferase reporter clones of indicated genes were purchased (CTTN, PIK3CD, AKT3, MYO9B, RAC1) (Switchgear Genomics). 3′UTR of NCAPG2 was cloned downstream of Renilla luciferase in psiCHECK2 (Promega) cut with XhoI using the In Fusion HD cloning kit (Clontech) following manufacturer's recommendations, followed by sequence verification. Primers used to amplify the NCAPG2 3′UTR were:

(SEQ ID NO: 22)

| | |
|---|---|
| Fwd: | TAGGCGATCGCTCGAGCCAAGCCAACATCTCCAGAC; |

(SEQ ID NO: 23)

| | |
|---|---|
| Rvs: | AATTCCCGGGCTCGAGGATGTTGTCATTGCTTTATTACTCA. |

293T cells were seeded in 96-well plates at 30,000 cells/well and incubated at 37° C. and 5% CO₂ for 16-24 hours. 293T cells were co-transfected with 200 ng 3′UTR reporter plasmid and 50 nM indicated mimic or control miRNA (Dharmacon) using Lipofectamine2000 (Invitrogen) following manufacturer's recommendations. Liposomal complexes of 3′UTR construct and miRNA mimic were prepared separately in 50 μL volumes, then added consecutively to appropriate wells of the 96-well plate. Cells were incubated at 37° C. and 5% CO₂ overnight. Media was aspirated from the wells and replaced with PBS. Luciferase assay was performed using Dual Glo Luciferase Assay kit (Promega—for NCAPG2) or Lightswitch Assay Reagent (Switchgear Genomics—for all others) following manufacturer's recommendations. Luminescence was measured in an Envision Multilabel plate reader (Perkin Elmer). Raw ratios of Renilla to Firefly luciferase (NCAPG2) or Renilla luciferase (Switchgear constructs) were normalized to empty vector and are relative to mock treatment (no transfection of miRNA mimic or control). Data represent average readings from replicate experiments (n>3). Data was plotted and significance determined in Graphpad Prism using 1-way ANOVA with Dunnett's multiple comparison post-testing using two different scrambled oligonucleotides as controls:

SCR#1 (Thermo Fisher Dharmacon miRIDIAN
microRNA Mimic Negative Control #1):
(SEQ ID NO: 24)
UCACAACCUCCUAGAAAGAGUAGA,
and
SCR#2 (Thermo Fisher Dharmacon miRIDIAN
microRNA Mimic Negative Control #2):
(SEQ ID NO: 25)
UUGUACUACACAAAAGUACUG.

Western Blotting.

Protein lysates were generated using RIPA buffer (Thermo Fisher) supplemented with protease inhibitors (Complete EDTA-free, Roche) and phosphatase inhibitors (PhosStop, Roche) for 20 minutes on ice, followed by centrifugation for 15 minutes at 13,000 rpm at 4° C. Protein-containing supernatant was transferred to fresh microcentrifuge tubes and stored below −20° C. until further use. Protein was quantified using DC Protein Assay (Biorad) following manufacturer's recommendations, with standard curves generated with bovine serum albumin (Sigma Aldrich). 40 μg or 10 μg (CTTN) of total protein lysate was loaded per lane of 4-20% Novex tris-glycine polyacrylamide mini gels (Invitrogen). SDS-PAGE was run at 150V for 1.5 to 2 hrs. Proteins were transferred to nitrocellulose or PVDF membranes using an iBlot semidry transfer system (Invitrogen) for 7 min on program 3. Membranes were washed 1× in diH₂O quickly followed by blocking with 5% non-fat dry milk (Carnation) in PBS for 60 minutes at room temperature. After blocking, membranes were washed briefly with PBS. Membranes were cut appropriately to examine multiple proteins per gel. Membranes were then incubated on a plate shaker overnight at 4° C. with primary antibodies diluted in Tris-buffered saline supplemented with 0.1% Tween-20 (TBS-T). Membranes were washed extensively with TBS-T (minimum 4× for 5 minutes), followed by incuation with appropriate horseradish peroxidase-conjugated secondary antibodies diluted in TBS-T+2% non-fat dry milk for 30-60 minutes at room temperature on a plate shaker. Membranes were washed extensively with TBS-T (minimum 4× for 5 minutes). Signal was detected using ECL Plus Chemiluminescent detection system (GE Healthcare) following manufacturer's recommendations. The following primary antibodies were used: NCAPG2 (Sigma Atlas), Tubulin (Sigma), CTTN (Millipore, clone 4F11), CDC42 (Cell Signaling, #2462), and PIK3CD (Santa Cruz, clone A-8). Secondary antibodies were HRP conjugated anti-mouse or rabbit IgG (GE Healthcare).

### Example 2

### miRNA Expression Profile Reveals Differential Expression Between Primary Melanoma Tumors which are Recurrent and Non-Recurrent

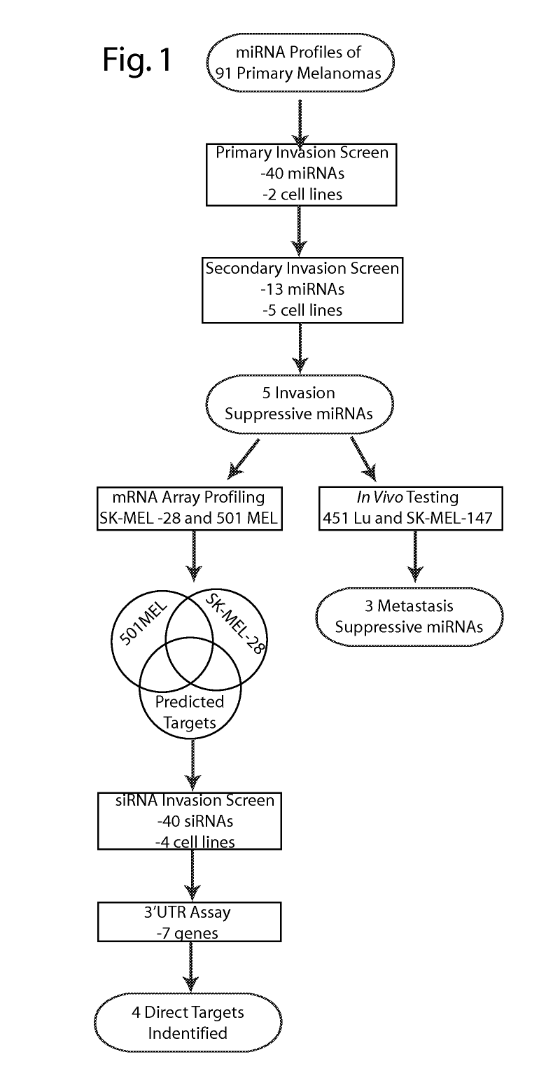

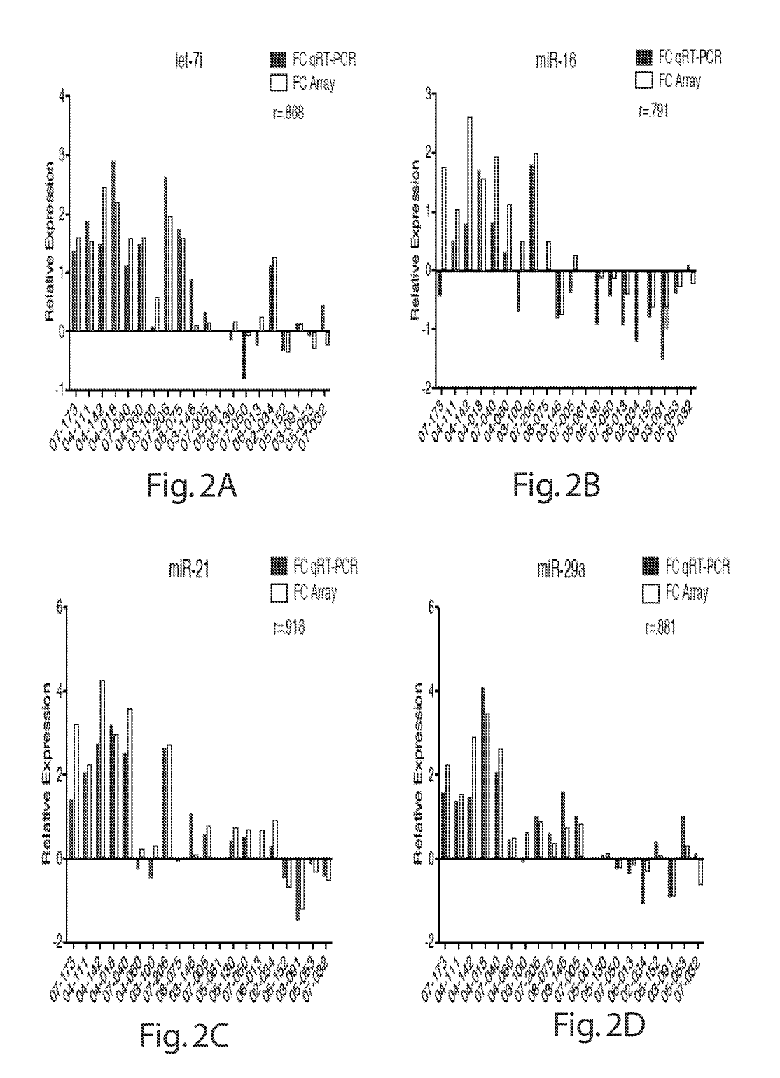

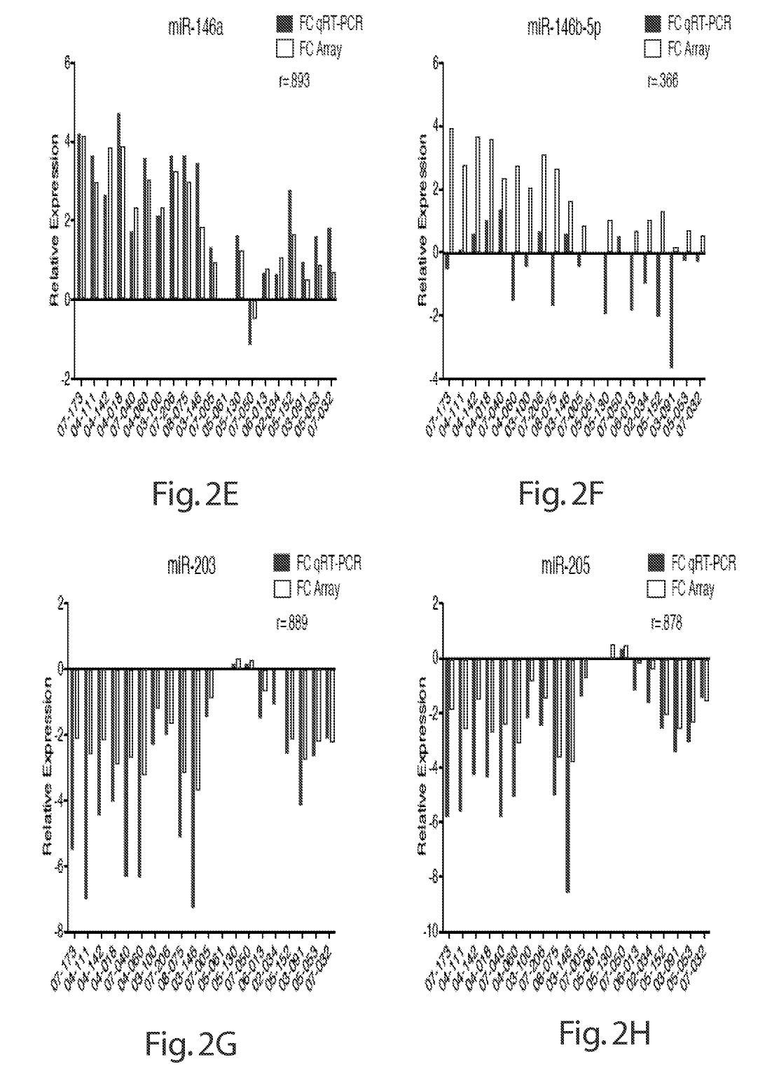

The miRNA expression was profiled by microarray of a well-annotated cohort of 92 primary melanomas with minimum patient follow-up of three years for surviving individuals to discover metastasis relevant miRNAs and develop predictive models of recurrence. miRNA expression profiling of FFPE (formalin fixed paraffin embedded)-extracted RNA from primary melanomas was performed. The quantified signals were background corrected (Normexp with offset value 10—Ritchie et al., Bioinformatics, 2007, 23(20):2700-2707) and normalized using the global Lowess (LOcally WEighted Scatterplot Smoothing) regression algorithm (Cleveland, W. S., 1979, J. Amer. Statist. Assoc., 74:829-836). FIG. 1 depicts the series of experiments performed to deduce the differential miRNA profile. The differentially expressed miRNAs between primary tumors that did and did not recur (3-year minimum follow-up) and between thick and thin lesions were identified. Table 1 details the clinical parameters, including recurrence, histological subtype, and tumor thickness of the 91 patients in the Discovery cohort. Randomly selected miRNAs were subsequently validated by quantitative real-time polymerase chain reaction (qRT-PCR). Eight such miRNAs were validated as shown in FIG. 2 to deem the array data robust. As surrogate clinical parameters of aggressive, invasive tumors, miRNAs differentially expressed between recurrent (n=46) and non-recurrent (n=45) patients and miRNAs whose expression correlated with tumor thickness were identified.

TABLE 1

| Discovery | | | Stage | | | | Histologic Subtype | | | Thickness (mm) | | Follow Up (months) | | Ulceration | |
|---|---|---|---|---|---|---|---|---|---|---|---|---|---|---|---|
| Cohort | n | % | I | II | III | IV | Nodular | SSM | Other | Median | Range | Median | Range | Yes | No |
| Congenital | 9 | | | | | | | | | | | | | | |
| Nevi | | | | | | | | | | | | | | | |
| All Cases | 91 | | | | | | | | | | | | | | |
| Recurrent | 46 | 50.5 | 5 | 23 | 18 | 0 | 34 | 7 | 5 | 3.0 | .85-30 | 44 | 10-219 | 29 | 17 |
| Non-recurrent | 45 | 49.5 | 14 | 29 | 2 | 0 | 24 | 18 | 3 | 2.2 | .9-11.0 | 75 | 31-103 | 17 | 28 |
| Stage I/II | 70 | | | | | | | | | | | | | | |
| with 3 yr | | | | | | | | | | | | | | | |
| Follow Up | | | | | | | | | | | | | | | |
| Recurrent | 28 | 39.4 | 5 | 23 | 0 | 0 | 20 | 5 | 3 | 2.79 | .85-10.1 | 60.5 | 18-219 | 19 | 9 |
| Non-recurrent | 42 | 60.6 | 14 | 28 | 0 | 0 | 22 | 17 | 3 | 2.2 | .9-11.0 | 75.5 | 37-103 | 17 | 25 |

### Example 3

### miRNAs Whose Expression is Lower in More Aggressive Primary Melanomas are Identified as Suppressors of In Vitro Invasion

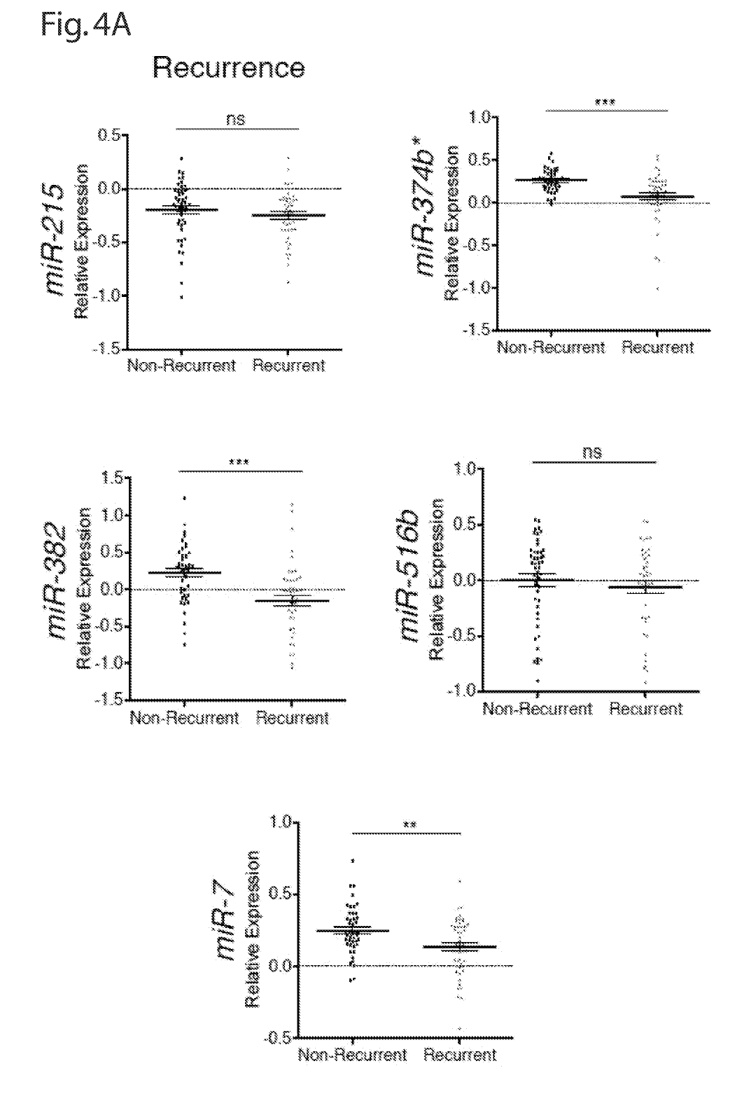

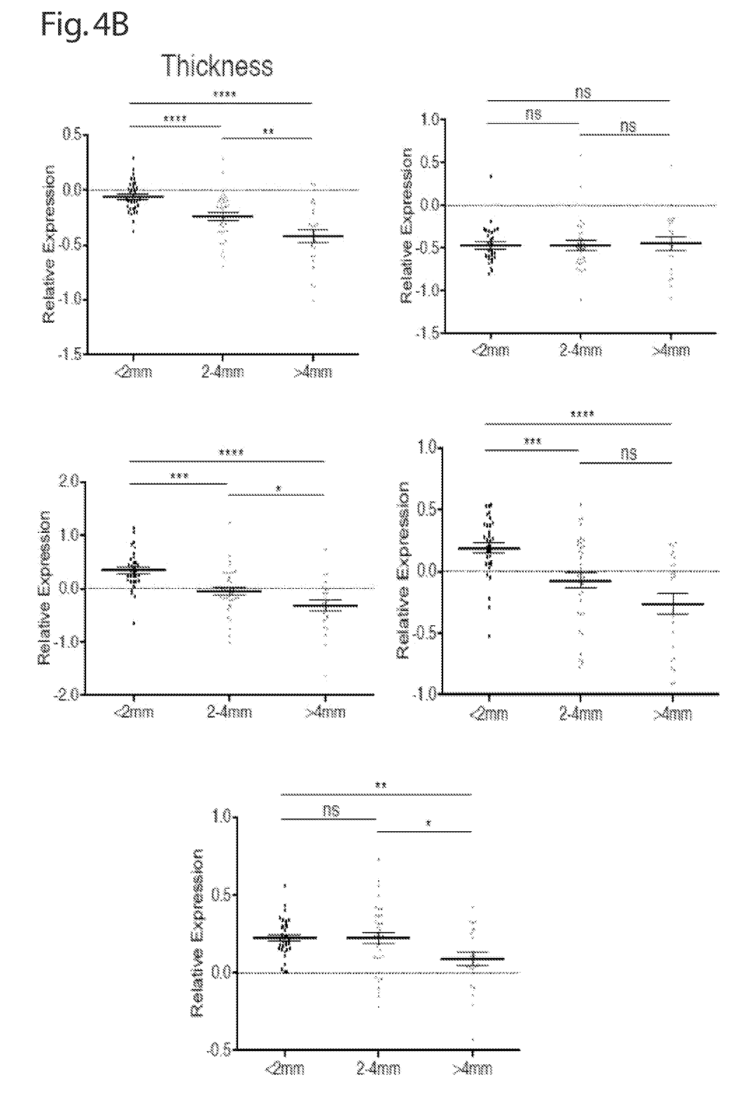

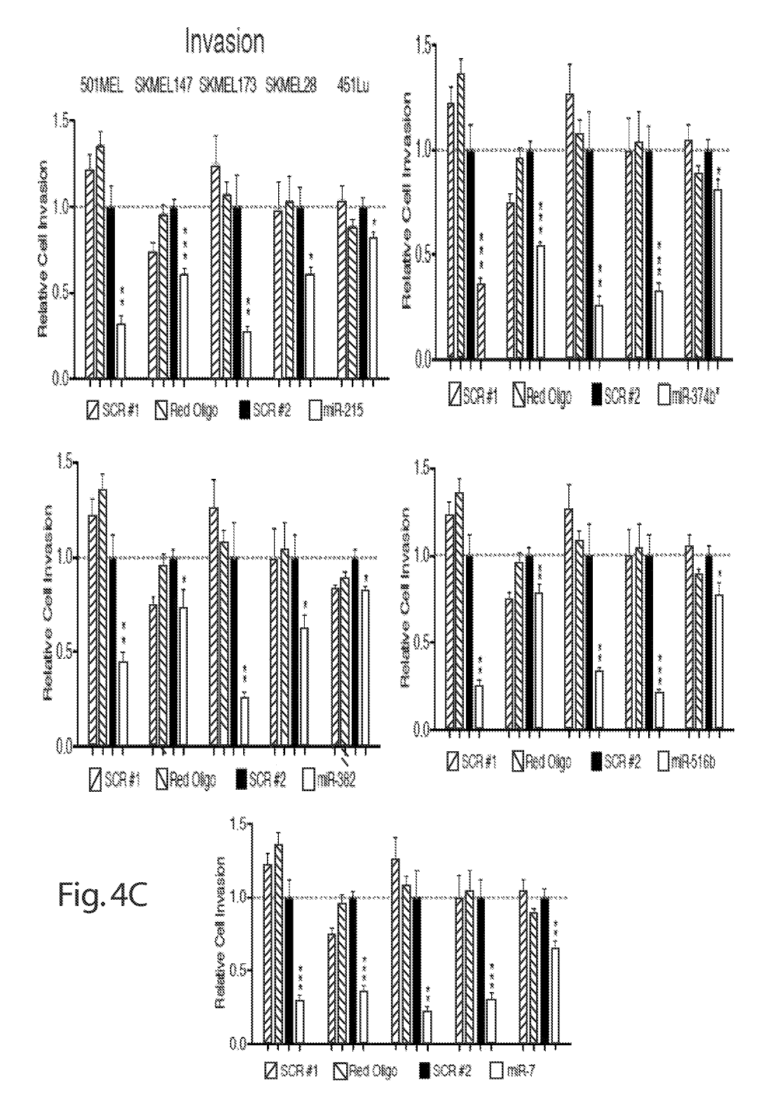

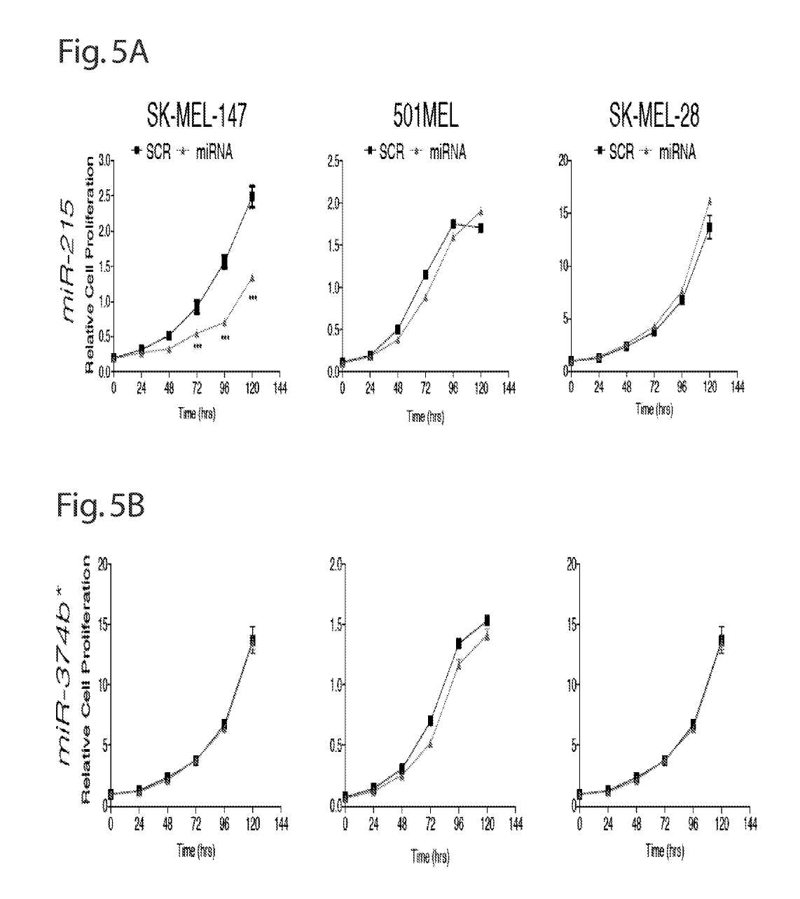

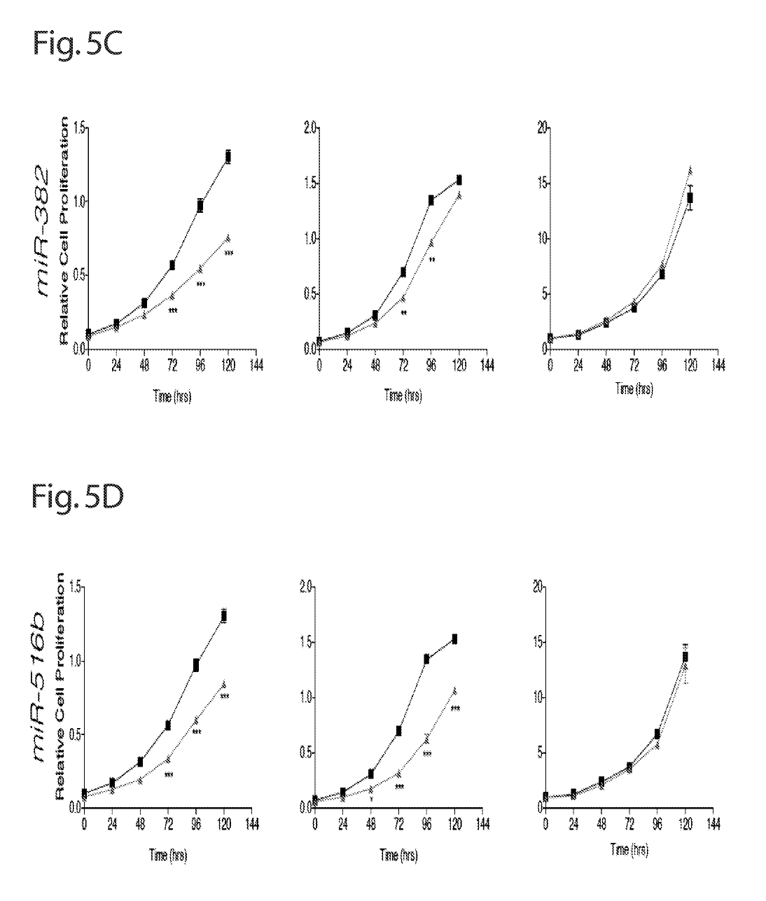

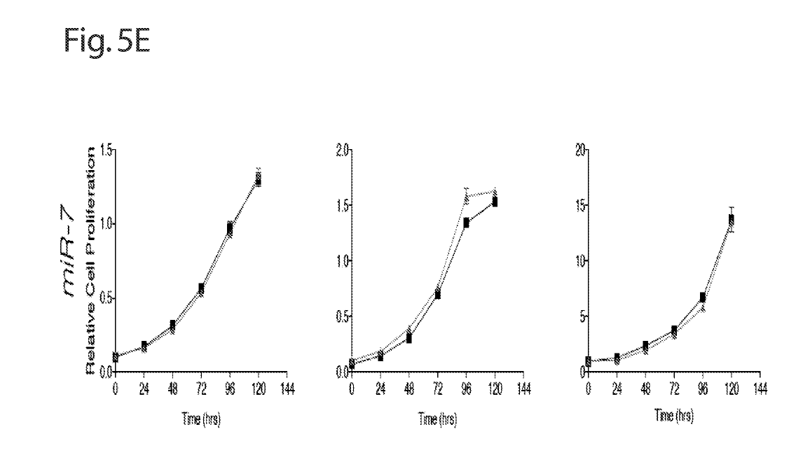

Forty candidates were selected from the array analyses and tested in a high-throughput in vitro invasion screen in two metastatic melanoma cell lines (501MEL, SK-MEL-147) to identify miRNAs that may functionally impact melanoma metastasis. Thirteen of these miRNAs were further screened across three additional melanoma cell lines (451Lu, SK-MEL-28, SK-MEL-173) in both in vitro invasion and cell proliferation assays. These analyses showed miR-215, miR-374b*, miR-382, miR-516b, and miR-7 as being less expressed in recurrent as compared to non-recurrent and/or thicker as compared to thinner tumors (FIG. 4A-B). Hence, these three miRNAs were identified as potent suppressors of in vitro invasion (FIG. 4C) in the melanoma cell lines tested. Ectopic expression of miR-7 or miR-374b* in melanoma cells had no impact on cell proliferation (FIG. 5) while miR-215 inhibited SK-MEL-147 proliferation; however, all three were potently suppressive of in vitro invasion (FIG. 4C). In contrast, miR-382 and miR-516b modestly suppressed proliferation of SK-MEL-147 and 501MEL but not SK-MEL-28, yet suppressed invasion in all three. These data suggest the invasion effects of the identified miRNAs are mostly proliferation-independent. Moreover, all invasion data were normalized to cell input to further control for cell proliferation and cell survival effects in invasion assay. miR-215 and miR-7 are well characterized to have tumor-suppressive activity in most cellular contexts studied (refs 22-28), but little is known of miR-374b*, miR-382, and miR-516b. In sum, the present data identify five miRNAs, whose expression is lower in more aggressive primary melanomas, as suppressors of in vitro invasion: miR-215, miR-374b*, miR-382, miR-516b, and miR-7.

### Example 4

### Identification of miRNAs which Suppress Metastasis In Vivo

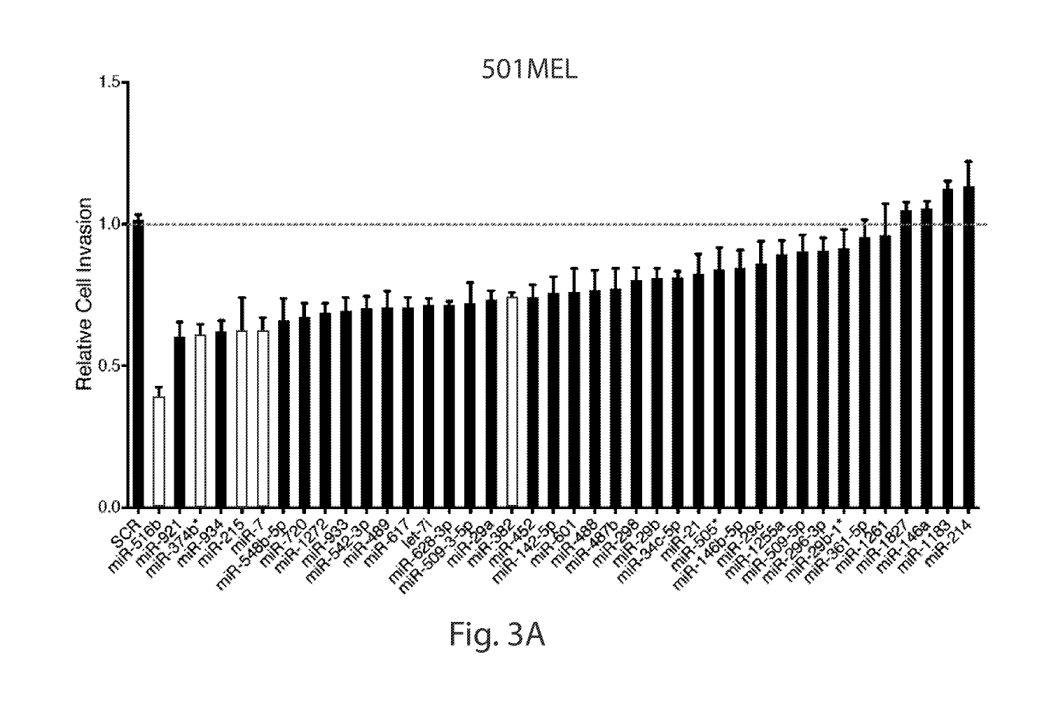

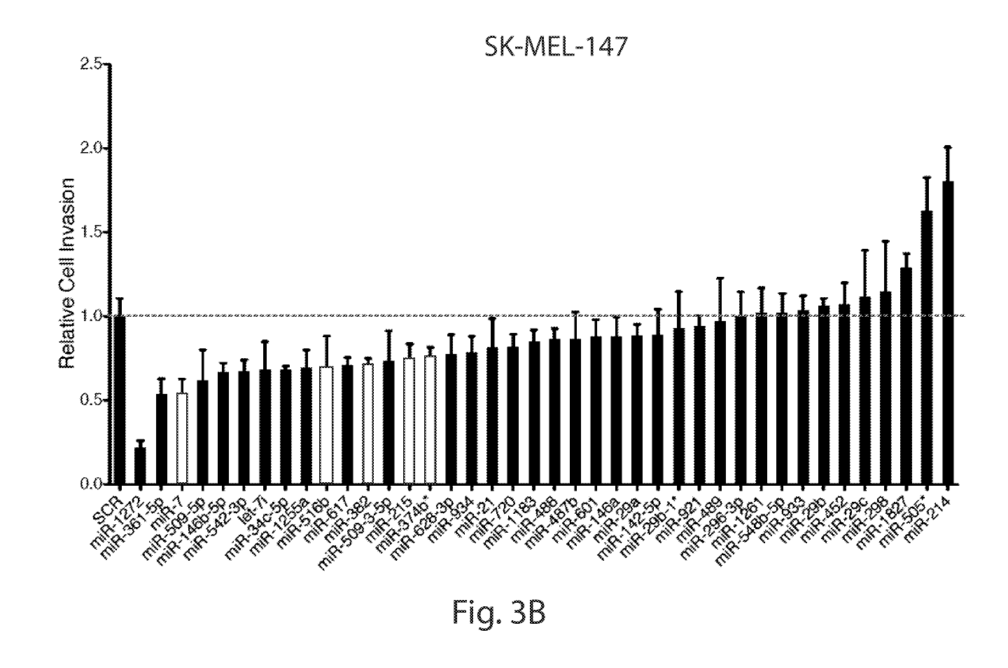

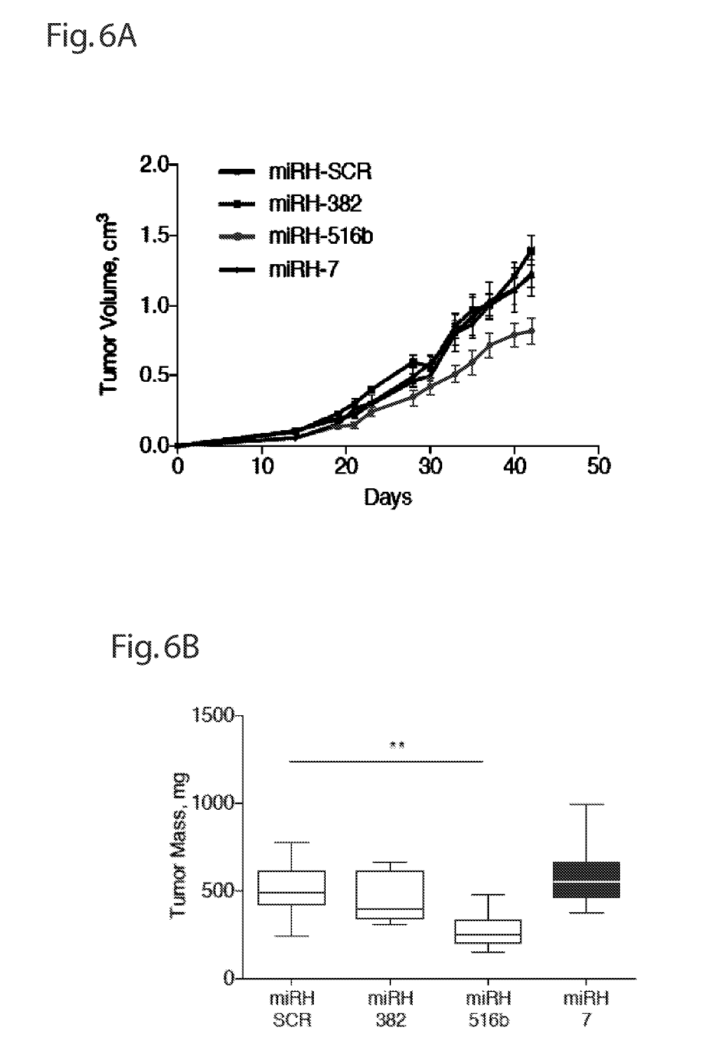

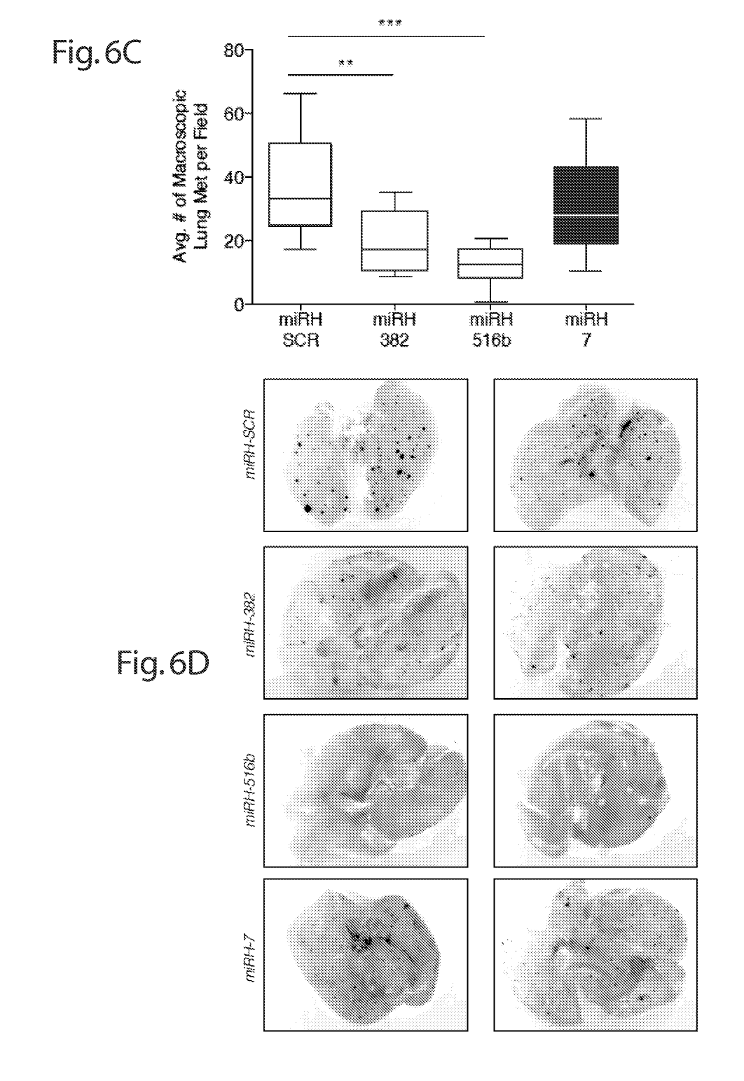

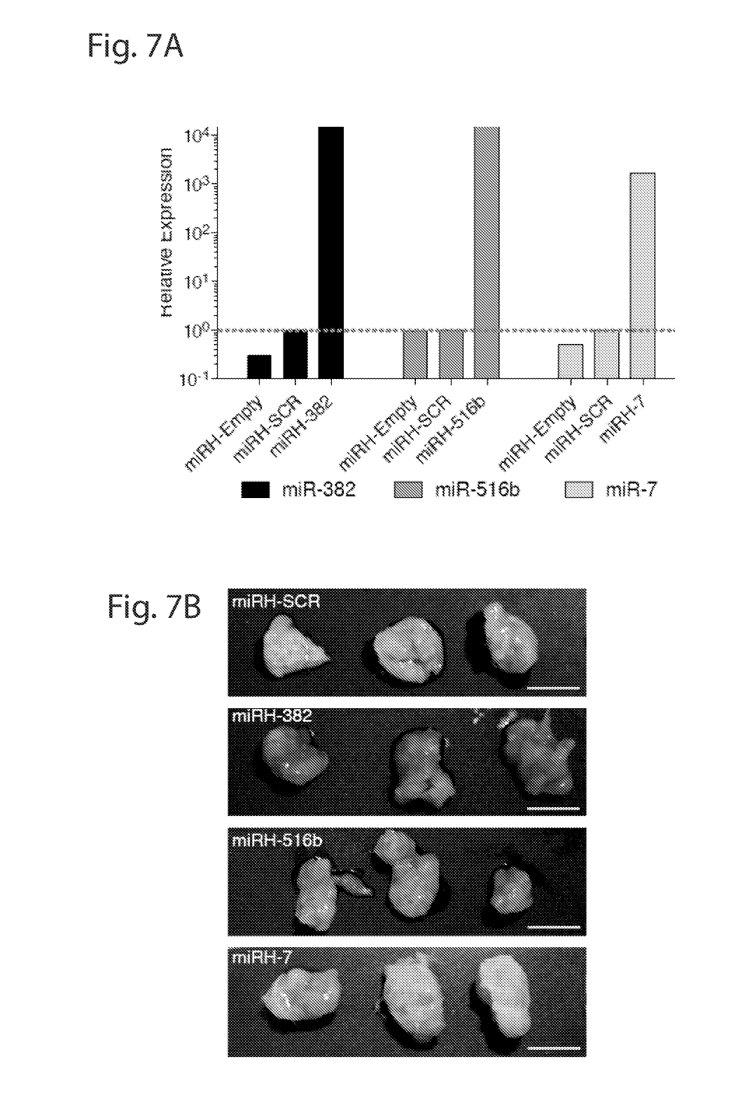

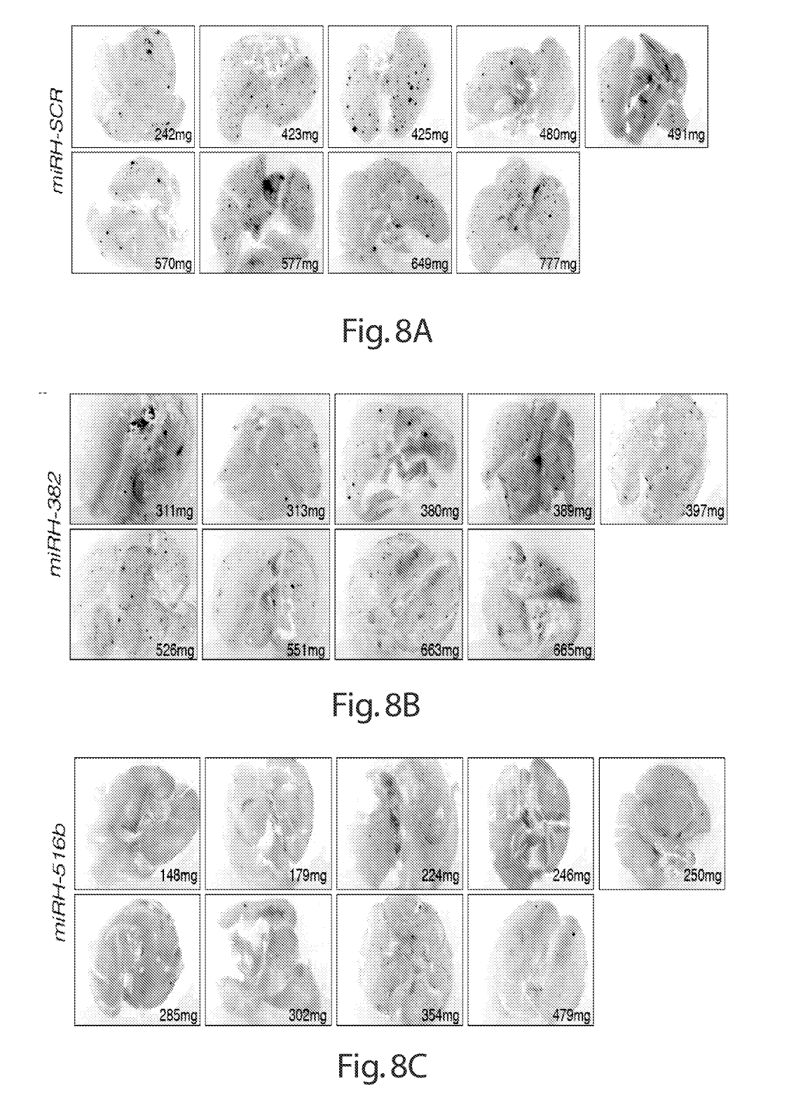

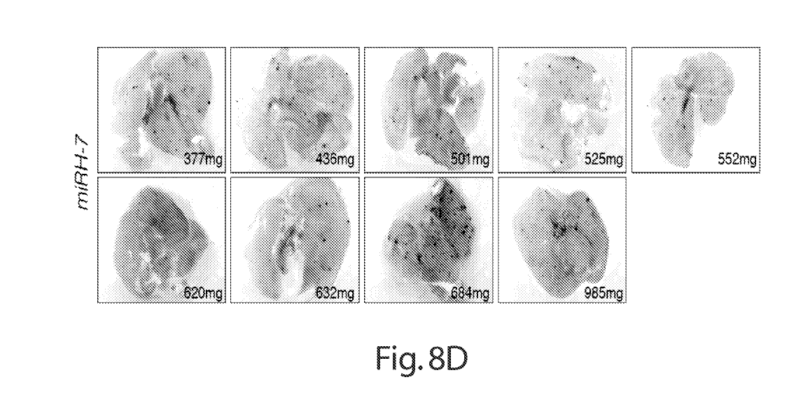

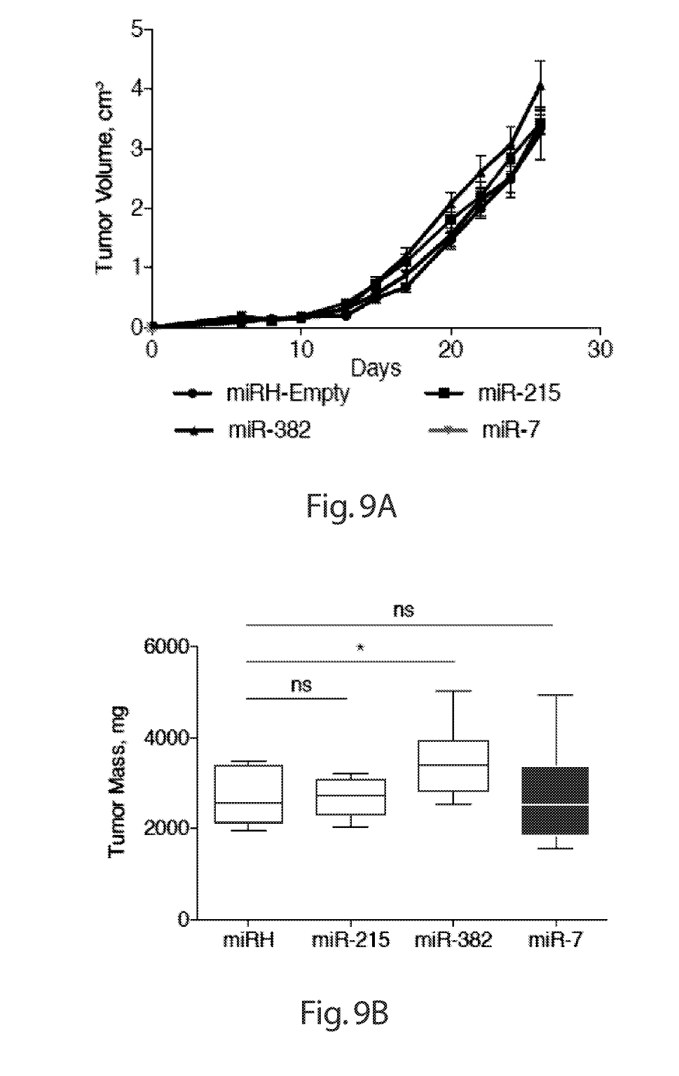

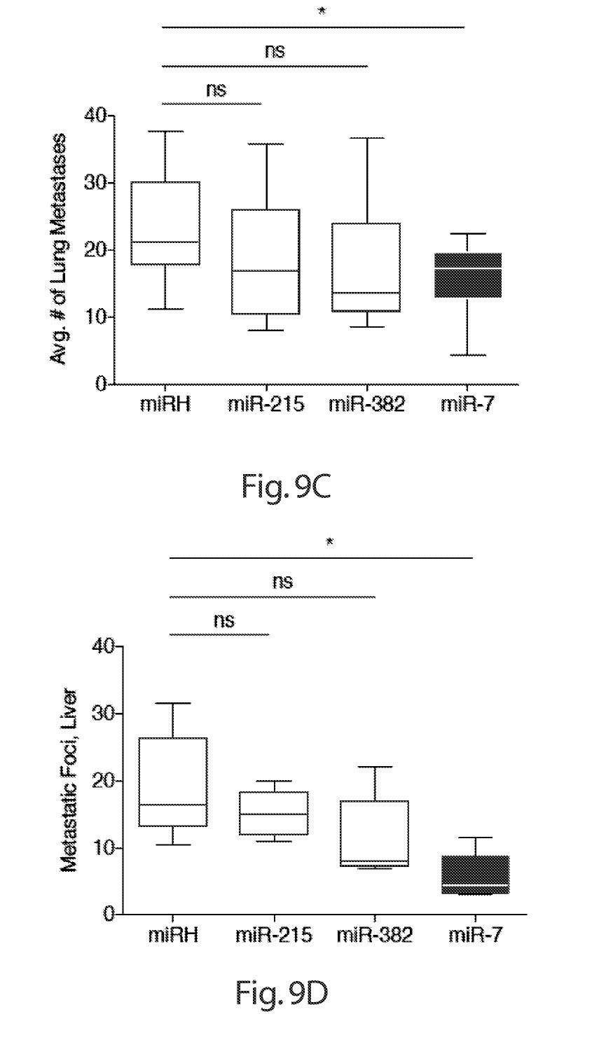

451Lu cells, which are highly metastatic to mouse lungs, were used to assess the impact of some of these miRNAs in vivo. miR-382, miR-516b, and miR-7 were ectopically expressed using lentiviral expression constructs containing the pre-miRNA and a GFP tracer to test their ability to suppress lung metastasis in mice. Primary tumor growth was unaffected by expression of miR-382 or miR-7, but was significantly decreased by miR-516b expression (FIG. 6A-B and FIG. 7). Importantly, fluorescence imaging of mouse lungs revealed striking reductions of metastatic foci in the lungs of mice with xenografts of miR-382 or miR-516b transduced cells (p=0.0047 or p=0.0002, respectively, relative to control (FIG. 6C-D, FIG. 8). Lungs of mice with miR-7-transduced cells had a similar number of macrometastases as control, however the size of these lesions appeared to be reduced, suggesting delayed metastasis or reduced proliferative capacity in the lungs (FIG. 6C-D). Moreover, in a separate experiment using SK-MEL-147 cells, miR-7 more clearly suppressed metastasis (FIG. 9). These data demonstrate that ectopic expression of miR-382, miR-516b, or miR-7 suppresses melanoma metastasis in vivo. Ectopic expression of miR-7 in melanoma cells had no impact on proliferation in vitro or in vivo, but was suppressive of invasion and metastasis (FIGS. 3, 4, 6, and 9), suggesting this as the dominant effect of miR-7 in melanoma. Table 2 lists the 40 miRNA candidates screened in the in vitro invasion assay and the fold change for each miRNA based on recurrent versus non-recurrent and thick versus thin tumors.

miR-516b, which also suppressed proliferation in several cell lines in vitro, was found to inhibit tumor growth in vivo. In addition, miR-516b potently suppressed lung metastasis in this model. The possibility that the effect of miR-516b on metastasis is a by-product of reduced tumor size cannot be excluded; however, there was minimal correlation of primary tumor size with metastatic burden (FIG. 7), suggesting reduced metastasis of miR-516b expressing tumors is, at least partly, independent of proliferation effects.

TABLE 2
miRNAs selected from miRNA array profile tested in in vitro invasion screen

| | | Correlation with Metastasis | | Correlation with Thickness | |
|---|---|---|---|---|---|
| miRNA ID | Hy3 Value | Log2 Fold Change | p value | Log2 Fold Change | p value |
| hsa-let-7i | 200 | 0.286023351 | 0.157 | 0.784549912 | 0.0001 |
| hsa-miR-1183 | 77 | −0.153491713 | 0.063 | −0.28052813 | 0.0005 |
| hsa-miR-1255a | 1147 | −0.209324148 | 0.024 | −0.173344023 | 0.0146 |
| hsa-miR-1261 | 646 | −0.196037854 | 0.032 | −0.22062581 | 0.0084 |
| hsa-miR-1272 | 113 | −0.180288163 | 0.041 | −0.372704402 | 0.0000 |
| hsa-miR-142-5p | 94 | −0.251808608 | 0.452 | −0.012206113 | 0.9616 |
| hsa-miR-146a | 333 | 0.451571419 | 0.173 | 1.211329258 | 0.0001 |
| hsa-miR-146b-5p | 369 | 0.403201911 | 0.190 | 1.169868747 | 0.0001 |
| hsa-miR-1827 | 2251 | −0.196719418 | 0.024 | −0.179761324 | 0.0088 |
| hsa-miR-21 | 559 | 0.352772482 | 0.200 | 0.989436584 | 0.0019 |
| hsa-miR-214 | 536 | −0.029647711 | 0.601 | −0.251976424 | 0.0001 |
| hsa-miR-215 | 88 | −0.048048739 | 0.345 | −0.251852493 | 0.0000 |
| hsa-miR-296-3p | 115 | −0.180270869 | 0.093 | −0.484526752 | 0.0001 |
| hsa-miR-298 | 90 | −0.137595959 | 0.093 | −0.265241593 | 0.0004 |
| hsa-miR-29a | 354 | 0.300700199 | 0.257 | 0.980306539 | 0.0001 |
| hsa-miR-29b | 278 | 0.322312033 | 0.209 | 0.980552492 | 0.0001 |
| hsa-miR-29b-1* | 125 | −0.308126202 | 0.024 | −0.382129161 | 0.0075 |
| hsa-miR-29c | 59 | 0.11423535 | 0.335 | 0.553186817 | 0.0005 |
| hsa-miR-34c-5p | 56 | −0.076191181 | 0.156 | −0.185443969 | 0.0005 |
| hsa-miR-361-5p | 138 | 0.053907606 | 0.521 | 0.241016527 | 0.0011 |
| hsa-miR-374b* | 95 | −0.192654752 | 0.010 | −0.115818929 | 0.0337 |
| hsa-miR-382 | 273 | −0.305623656 | 0.012 | −0.492625831 | 0.0000 |
| hsa-miR-452 | 108 | −0.06853463 | 0.370 | −0.302514556 | 0.0001 |
| hsa-miR-487b | 142 | 0.047851447 | 0.645 | 0.491928467 | 0.0032 |
| hsa-miR-488 | 144 | −0.091573848 | 0.046 | −0.153016823 | 0.0005 |
| hsa-miR-489 | 308 | −0.051599936 | 0.575 | −0.317090111 | 0.0001 |
| hsa-miR-505* | 200 | −0.19377753 | 0.024 | −0.265683122 | 0.0002 |
| hsa-miR-509-5p | 707 | −0.049965807 | 0.346 | −0.365055585 | 0.0000 |
| hsa-miR-509-3-5p | 487 | 0.199793079 | 0.060 | −0.011435083 | 0.9190 |
| hsa-miR-516b | 361 | −0.060010761 | 0.293 | −0.339017819 | 0.0001 |
| hsa-miR-542-3p | 280 | −0.134955223 | 0.101 | −0.325297884 | 0.0000 |
| hsa-miR-548b-5p | 99 | −0.373200429 | 0.041 | −0.738677442 | 0.0002 |
| hsa-miR-601 | 105 | −0.190678103 | 0.033 | −0.145792438 | 0.0700 |
| hsa-miR-617 | 464 | −0.104318661 | 0.239 | −0.496227812 | 0.0001 |
| hsa-miR-628-3p | 3704 | −0.043894125 | 0.480 | −0.199187963 | 0.0005 |
| hsa-miR-7 | 129 | −0.120867369 | 0.051 | −0.055064373 | 0.2453 |
| hsa-miR-720 | 14784 | 0.19270498 | 0.351 | 0.74748153 | 0.0005 |
| hsa-miR-921 | 478 | −0.259246691 | 0.058 | −0.412152677 | 0.0002 |
| hsa-miR-933 | 1871 | 0.061943317 | 0.553 | 0.352372534 | 0.0005 |
| hsa-miR-934 | 162 | −0.138366596 | 0.041 | −0.149350472 | 0.0075 |

### Example 5

### Metastasis-Suppressive miRNAs Directly Target mRNAs Whose Depletion Inhibits Invasion

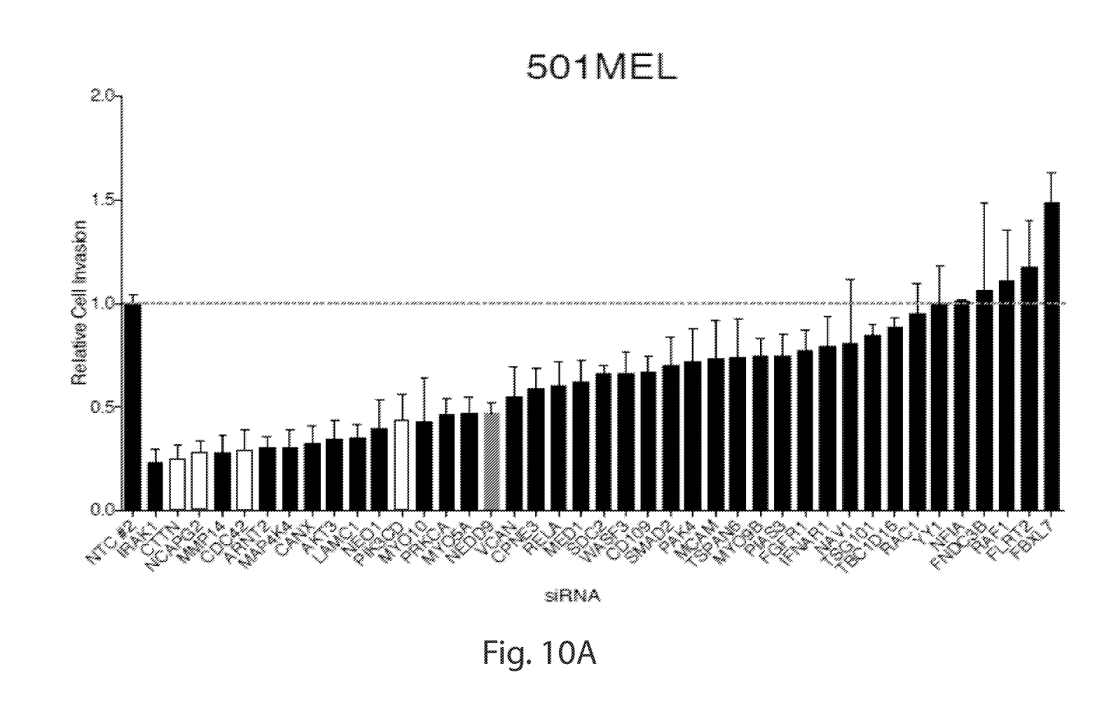

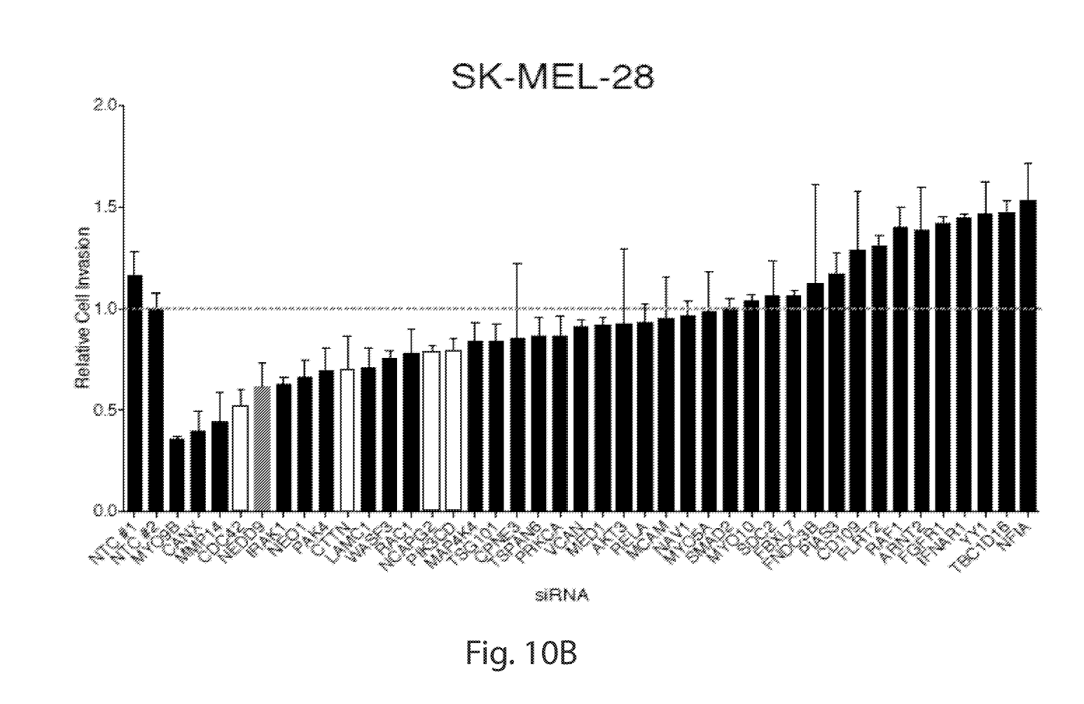

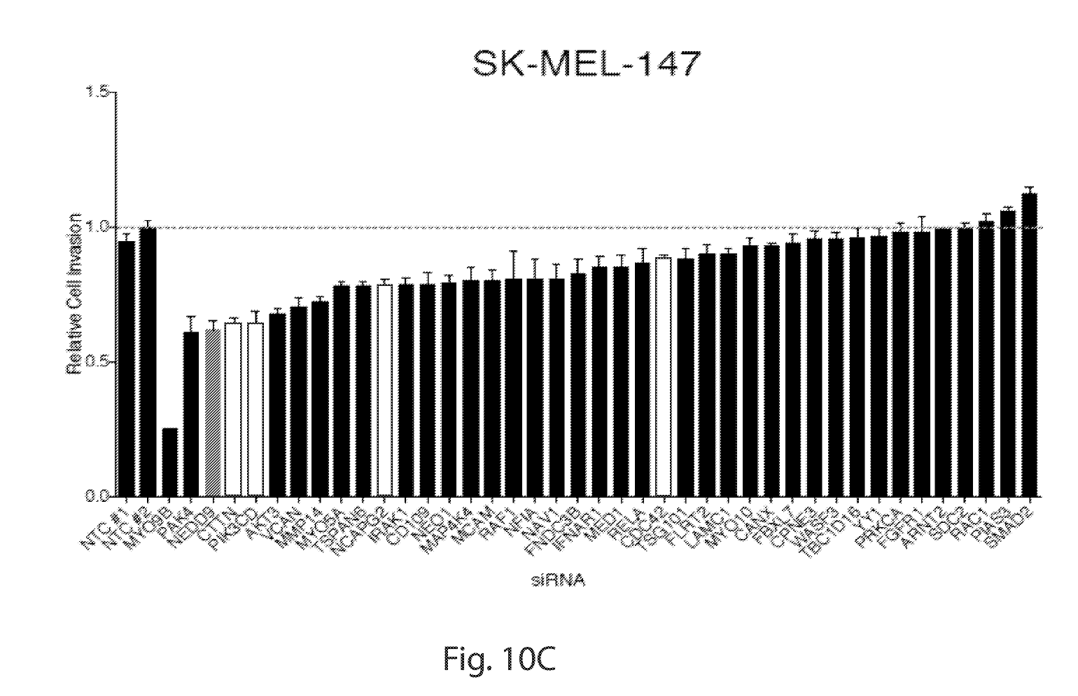

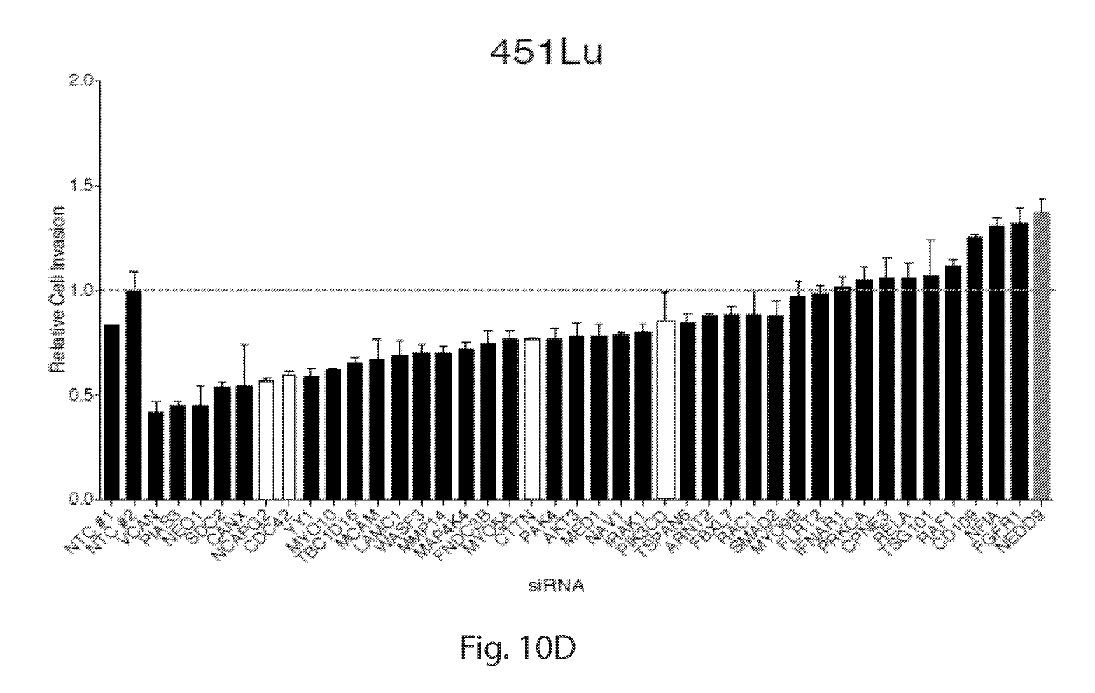

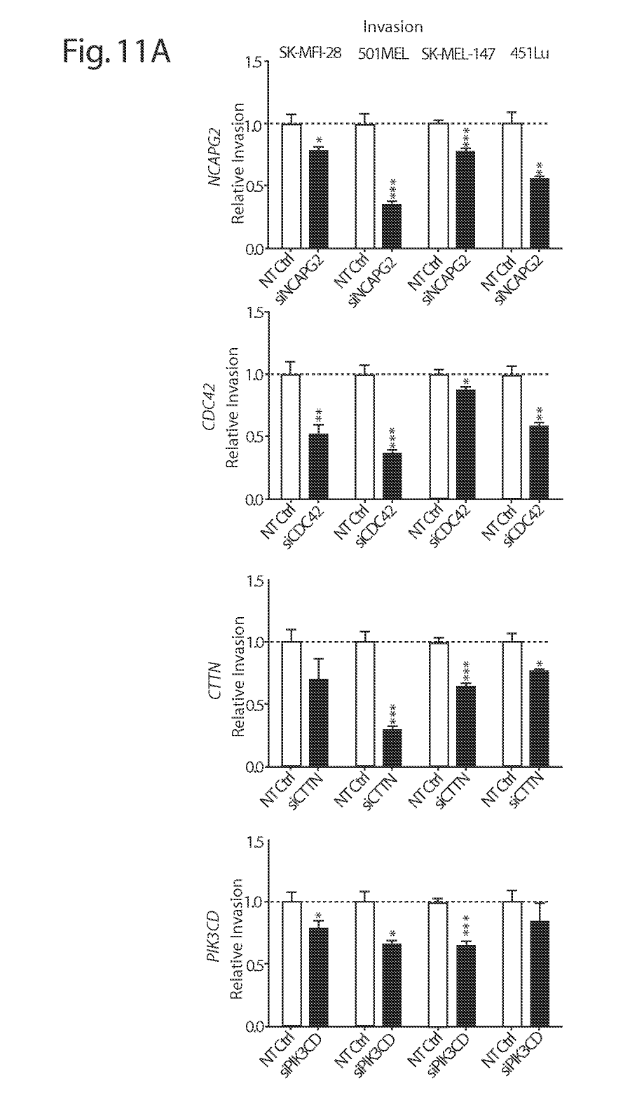

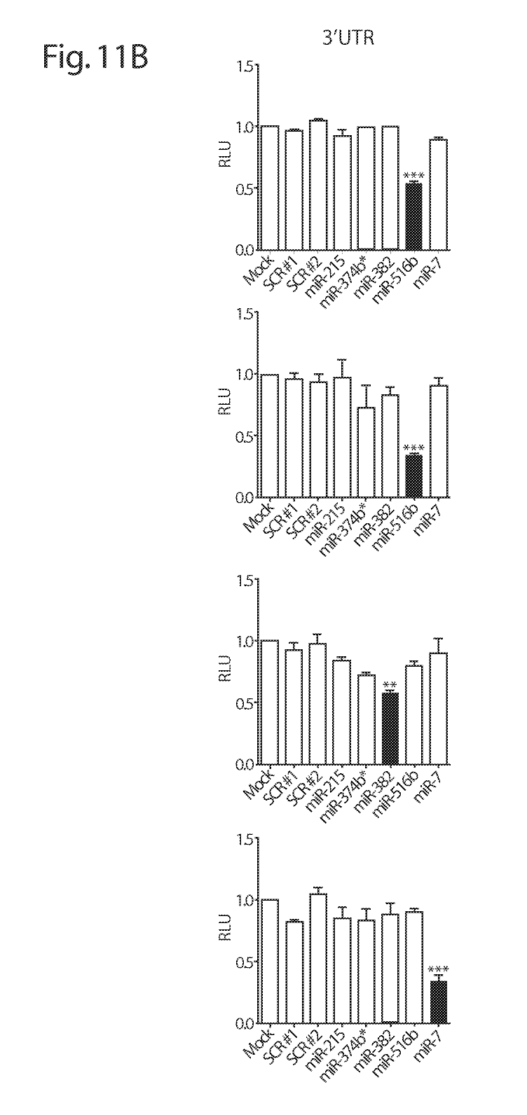

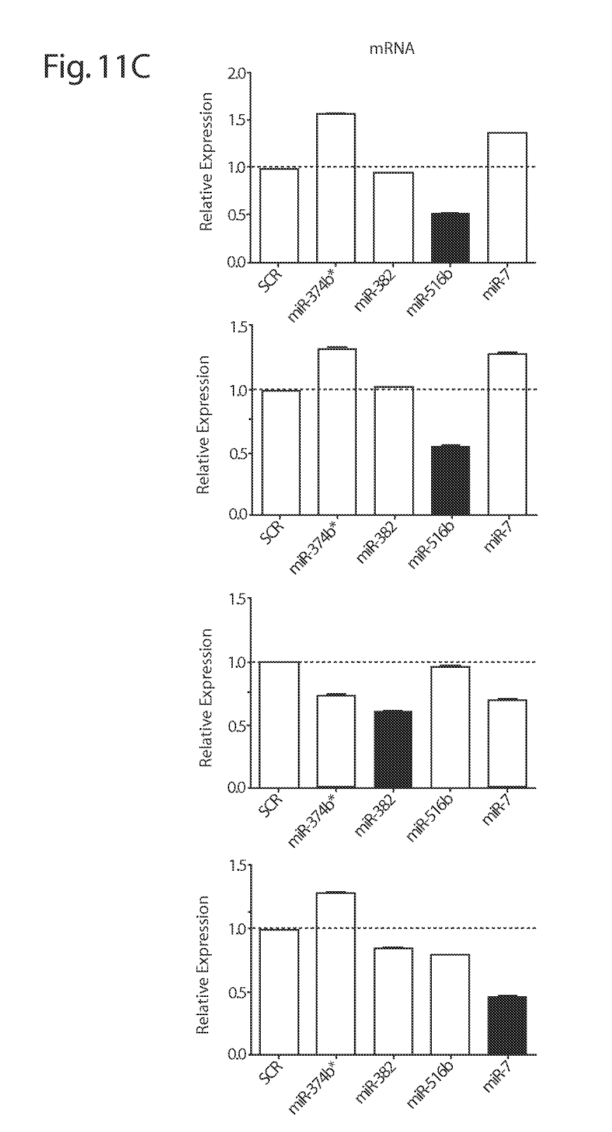

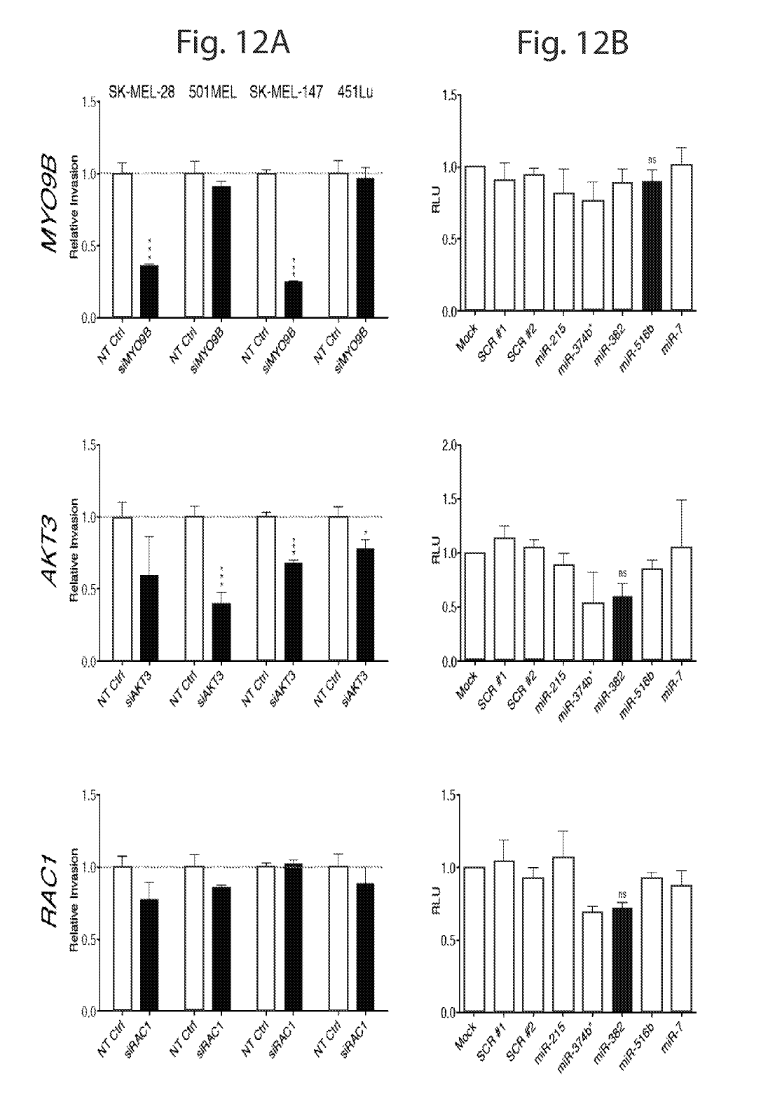

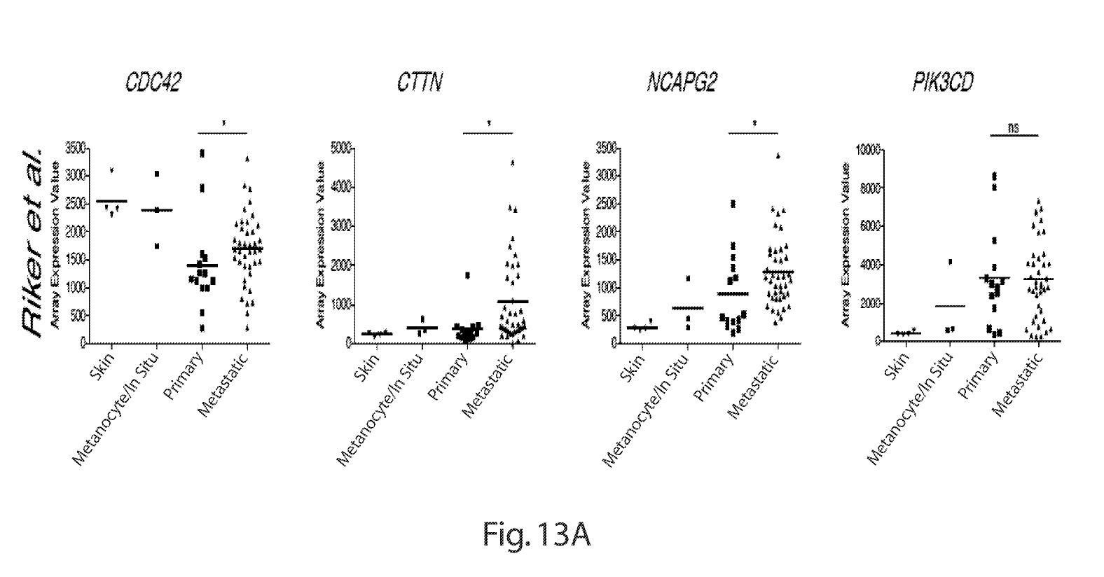

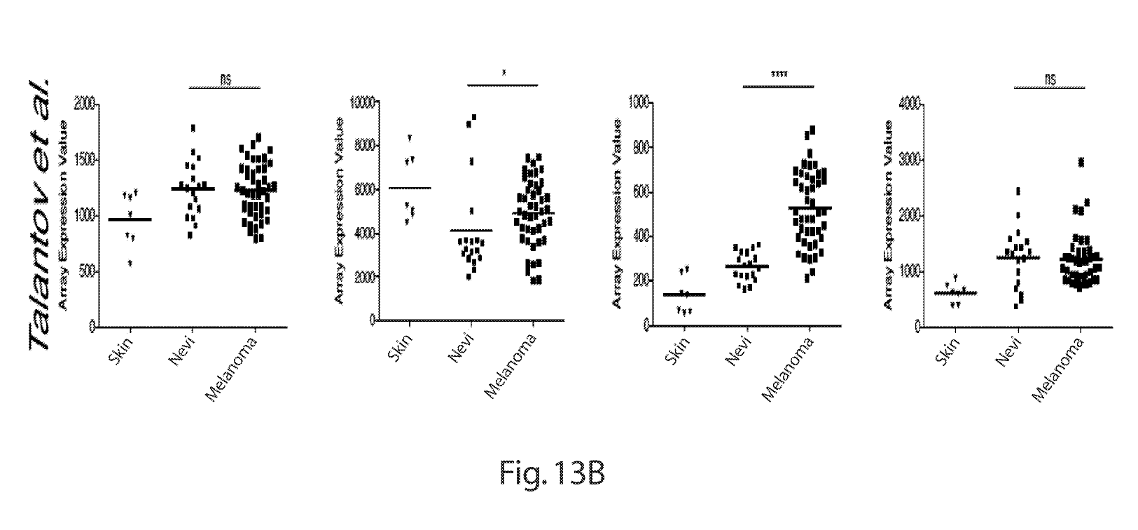

To better understand the mechanisms by which miR-382, miR-516b, and miR-7 function to suppress invasion and metastasis, the present inventors sought to identify direct targets that could mediate antimetastatic phenotype. Potential downstream mediators of these miRNAs were selected by mRNA array analysis and tested in a secondary invasion screen. mRNA expression array analysis of two melanoma cell lines (SK-MEL-28 and 501MEL) over-expressing a control or individual invasion-suppressive miRNA was performed. Transcripts downregulated by each specific miRNA relative to scrambled control were identified in both cell lines and overlapped this list with that of the miRNA's predicted targets (Targetscan v5.2 [Targetscan] or miRANDA [http://www.microrna.org]) and Clip-seq reads mapped to predicted target binding sites (starbase.sysu.edu.cn) (refs 29-33). 40 candidate genes were selected from the resulting lists. These candidates were tested in this automated in vitro invasion assay by siRNA-mediated depletion in four melanoma cell lines to identify putative miRNA targets whose silencing could also suppress invasion (FIG. 10). As expected, the data showed that depletion of many of these candidate genes results in suppression of invasion. Seven genes were selected (NCAPG2, CTTN, CDC42, RAC1, AKT3, MYO9B, PIK3CD), whose effects on invasion were consistent in at least 3 of 4 cell lines screened, and tested whether they are direct miRNA targets by 3′UTR luciferase reporter assays (FIG. 11 and FIG. 12). As shown in FIG. 11A, 4 genes (NCAPG2, CDC42, CTTN, and PIK3CD) were identified whose depletion suppressed invasion in the four cell lines tested. Moreover, the 3′UTRs of these genes show clear regulation by the predicted targeting miRNA (FIG. 11B), suggesting that these may be key direct mediators of the invasion and metastasis-suppressing effects of miR-382, miR-516b, and miR-7. Direct targets were further confirmed by mRNA analysis (FIG. 11C). Moreover, in published data sets of melanoma expression profiling, CTTN and NCAPG2 levels were found increased in nevi vs melanoma or in metastatic melanoma vs primary melanoma providing independent support for the importance of these genes in disease progression (FIG. 13) (ref 34,35). Additionally, a recent study identified NCAPG2 as part of a gene signature of melanoma progression (ref 36). Despite modulation by microRNA overexpression in cell lines and siRNA-mediated inhibition of invasion, AKT3, RAC1, and MYO9B were not consistently identified as direct targets of the miRNAs tested. These candidate targets may act as indirect downstream effectors or may be entirely independent. In summary, several direct targets of metastasis-suppressive microRNAs have been identified whose depletion recapitulates invasion repression.

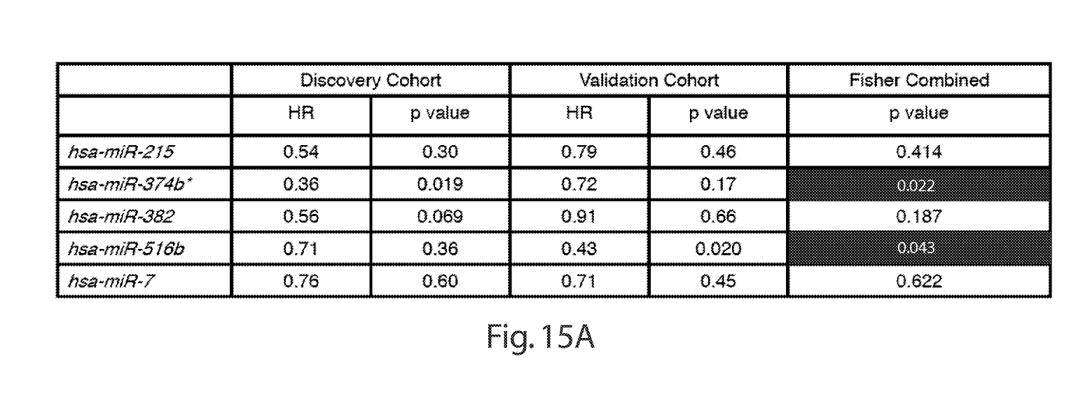

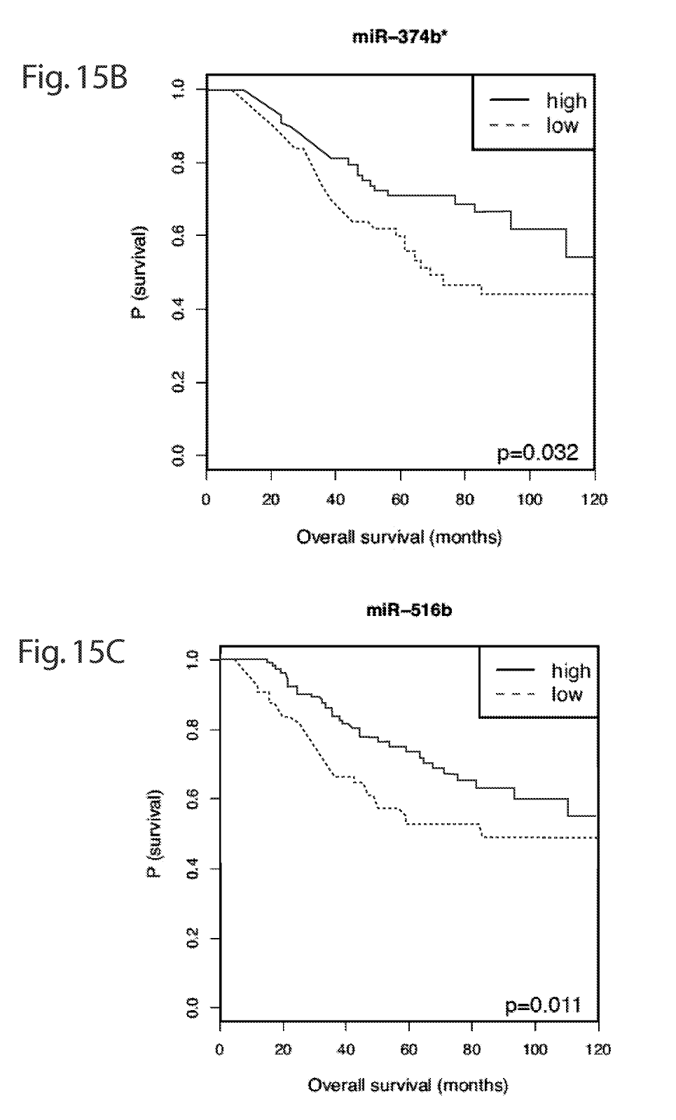

A panel of cell lines was tested to ensure effects were applicable to most, if not all, melanomas. Analyses identified five miRNAs (miR-215, miR-374b*, miR-382, miR-516b, and miR-7) that consistently repressed invasion. Of the five miRNAs identified, evidence that three (miR-382, miR-516b, and miR-7) are suppressors of metastasis in vivo was shown. Further, analysis of the clinical data showed miR-374b*, miR-382, miR-516b expression independently correlates with overall survival of these patients, highlighting their importance in melanoma progression (FIG. 15).

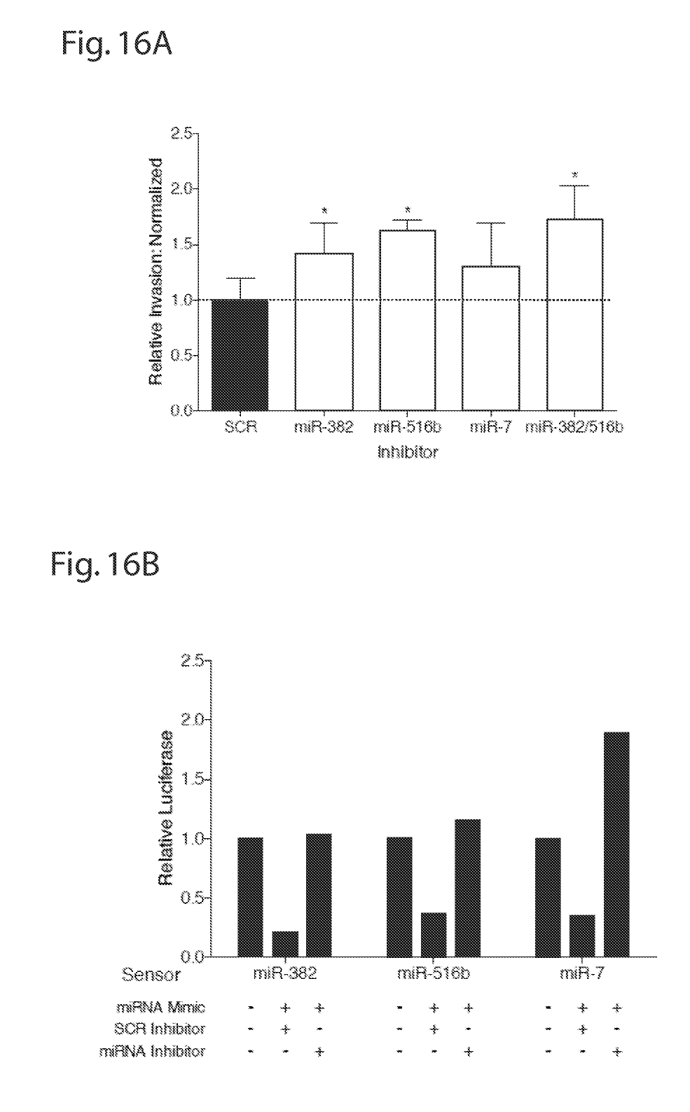

Further, the miRNAs identified have lower expression in aggressive primary tumors; thus in order to more closely recapitulate what occurs in the primary tumor, miR-382, miR-516b, and miR-7 were inhibited in a poorly invasive cell line to probe for effects on invasion. Inhibition of miR-382 and miR-516b alone or in combination enhanced the invasive capacity of these cells, further supporting the biological relevance of the present findings (FIG. 16).

### Example 6

### Development of Predictive Models of Recurrence

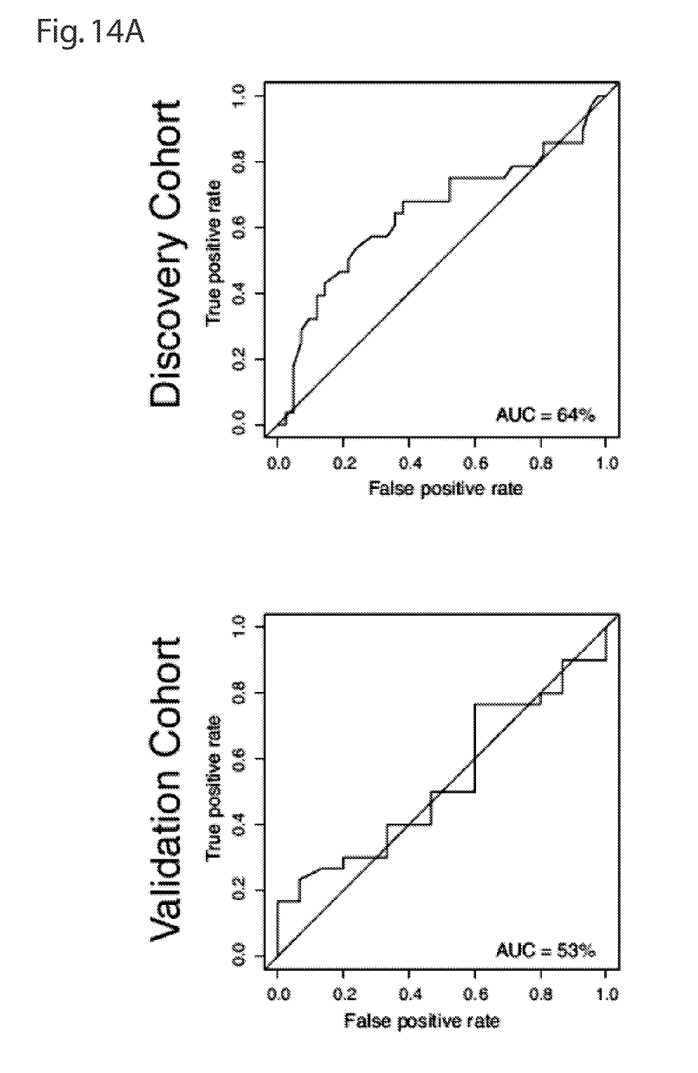

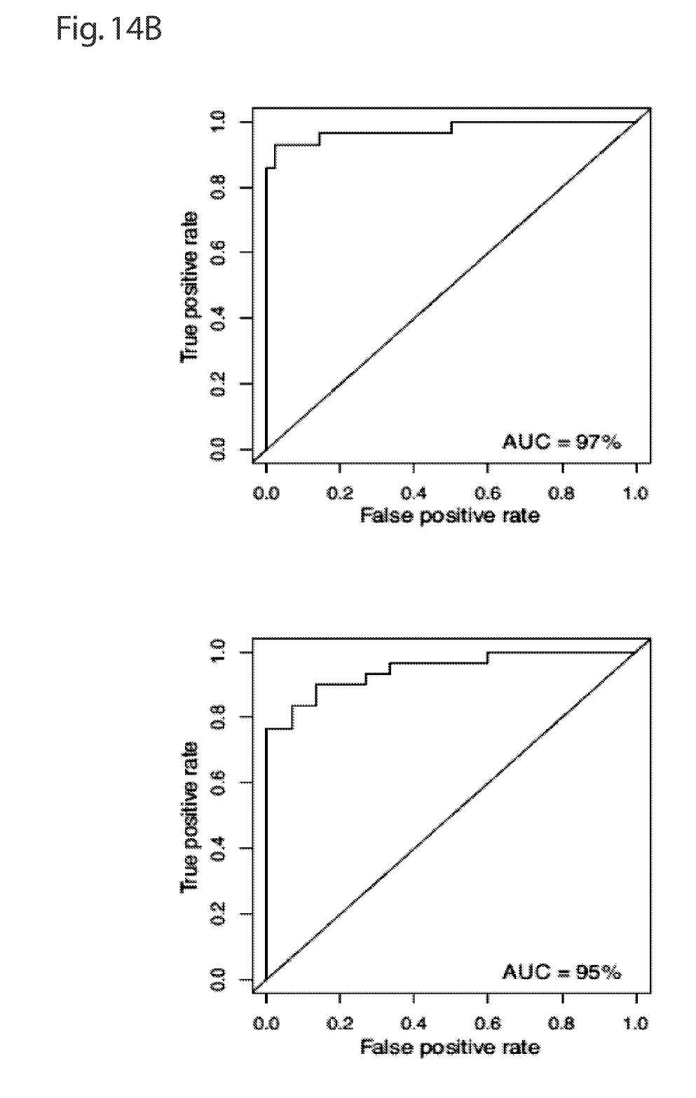

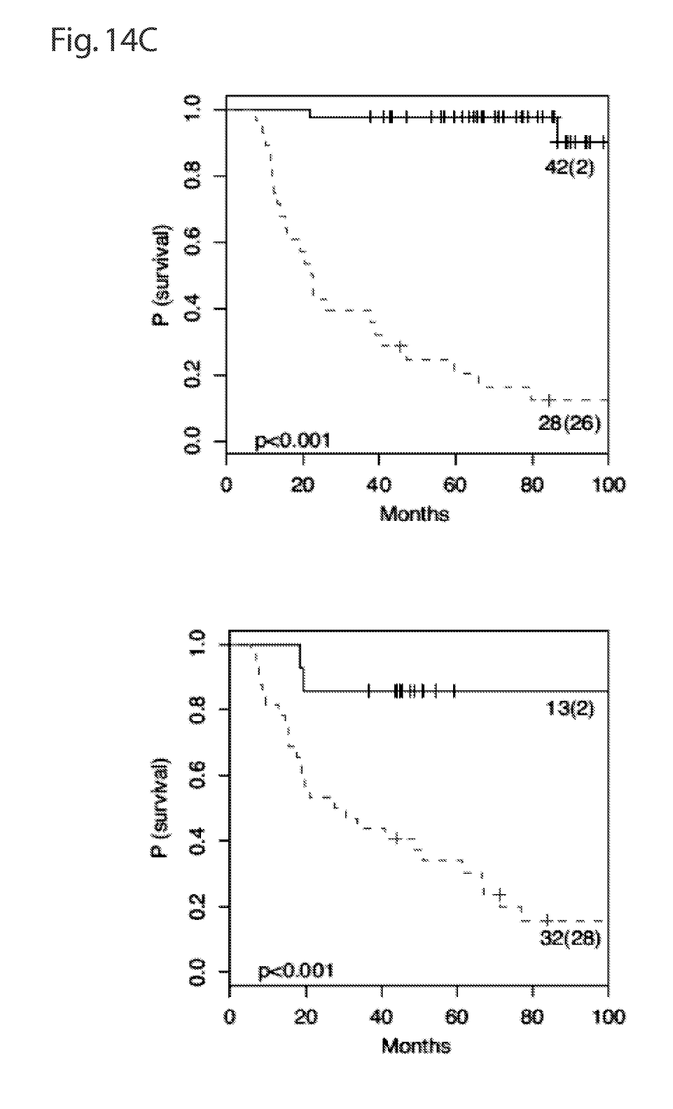

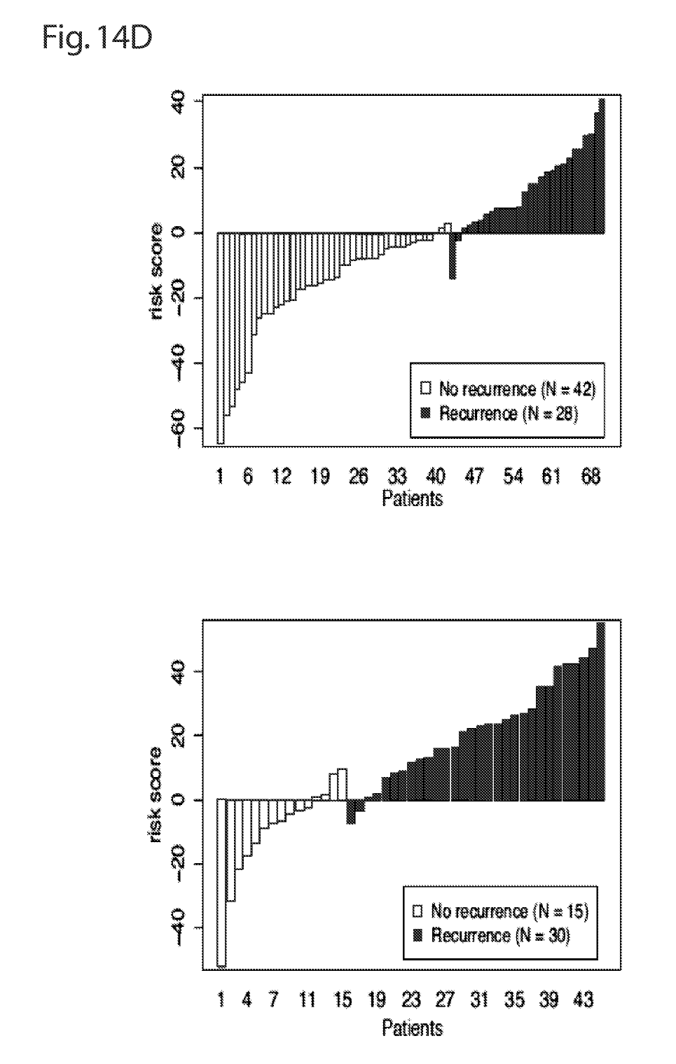

Finding a signature to robustly and accurately classify early stage patients by risk of disease progression is of great clinical importance. In order to address this question from the microRNA expression profile of 91 primary melanomas (discovery cohort), prognostic models of recurrence for stage I/II patients were developed. Risk models were developed using the 70 stage I/II patients (28 recurred, 42 not recurred) with at least three years of follow-up present in this cohort. Prognostic models using only clinical variables showed that the best, which included stage, thickness, and ulceration, had an AUC=64% under the receiver operating characteristic (ROC) curve with none of the predictors significant (FIG. 14A). A signature set was identified containing 21 miRNAs, including hsa-miR-10a, hsa-miR-1285, hsa-miR-374b*, hsa-miR-377*, hsa-miR-513b, hsa-miR-342-3p, hsa-miR-625*, SNORD3A, hsa-miR-1204, hsa-miR-574-3p, hsa-let-7a-2*, hsa-miR-615-3p, hsa-miR-564, hsa-miR-154*, hsa-miR-7, hsa-miR-215, hsa-miR-382, hsa-miR-663, hsa-miR-516b, hsa-miR-99b, and hsa-miR-1276. A risk score was obtained from the linear combination of predictors in the multiple logistic regression models resulting in a classifier with AUC=97%, 95% CI: (0.93, 1) (FIG. 14B). Using Youden's index of the ROC as a cut-off point to classify patients into high and low risk groups (Sensitivity=0.93, Specificity=0.95), the Kaplan-Meier curve shows that the two groups have a dramatic separation in recurrence-free survival (RFS) (FIG. 14C).

### Example 7

### Confirmation of Model Via an Independent Validation Cohort

To validate the described model, miRNA expression of an independent cohort of primary melanomas (n=113) was profiled including 69 stage I and II tumors, of which 30 patients recurred while 39 patients have not recurred and 15 of the 39 have at least 3 years of follow-up (Table 3). Applying the classifier developed using the discovery cohort to predict risk for recurrence in this validation cohort yielded an AUC=95%, 95% CI: (0.88, 0.99) of the ROC curve (FIG. 14B). Using the same threshold from the discovery cohort as a cut-off point to divide the validation cohort patients into high and low risk groups (sensitivity=0.93, specificity=0.73), the Kaplan-Meier curve shows the two groups again have a remarkable separation in RFS (FIG. 14C). In comparison, when predictive models using only clinical variables were explored in the validation cohort, the best, which used stage, thickness and ulceration, had an AUC of only 53% under the ROC curve (FIG. 14A). In conclusion, a robust tissue-based signature of 21 microRNAs, detectable from FFPE tissues, that can predict recurrence at the time of diagnosis using tissue specimens from 115 (70 for discovery and 45 for validation) stage I/II patients with extensive follow up was identified. Importantly, this signature includes miRNAs (miR-7, miR-382, miR-516b, miR-374b*, and miR-215) that were found to experimentally modulate melanoma invasion in vitro and metastasis in vivo, showing that some of these miRNAs are not just biomarkers of disease outcome but functionally influence it.

TABLE 3
Clinicopathological characteristic of patient samples in validation cohort

| Validation | | | Stage | | | | Histologic Subtype | | | Thickness (mm) | | Follow Up (days) | | Ulceration | |
|---|---|---|---|---|---|---|---|---|---|---|---|---|---|---|---|
| Cohort | n | % | I | II | III | IV | Nodular | SSM | Other | Median | Range | Median | Range | Yes | No |
| All Cases | 113 | | | | | | | | | | | | | | |
| Recurrent | 60 | 53.1 | 8 | 22 | 28 | 2 | 32 | 16 | 12 | 4 | .52-30 | 35 | 5-150 | 35 | 25 |
| Non-recurrent | 53 | 46.9 | 16 | 23 | 14 | 0 | 26 | 22 | 5 | 2.5 | .85-24 | 33 | 8-111 | 23 | 30 |
| Stage I/II | | | | | | | | | | | | | | | |
| with 3 yr | | | | | | | | | | | | | | | |
| Follow Up | | | | | | | | | | | | | | | |
| Recurrent | 30 | 66.7 | 8 | 22 | 0 | 0 | 13 | 10 | 7 | 2.95 | .52-12 | 37.5 | 13-150 | 15 | 15 |
| Non-recurrent | 15 | 33.3 | 4 | 11 | 0 | 0 | 7 | 6 | 2 | 4.5 | .85-12 | 50 | 36-101 | 8 | 7 |

### Example 8

### Development of the Four Logistic Regression Models for Prediction of Recurrence Risk Using the Discovery Cohort

FIG. 17 shows the area under the ROC of the discovery cohort and the validation cohort deduced from four logistic regression models. Tables 4 (a)-(d) outline the top ranking miRNA which were used to develop the four logistic regression models for prediction of recurrence risk using the discovery cohort. Note that, due to the software generated these tables, in these Tables 4(a)-4(d), the * at the end of the miRs is replaced by a period. For example has-miR-374b* becomes “has.miR.374b.” in Table 4(a).

TABLE 4a
Univariate logistic regression of 3-year
recurrence, with adjustment of stage

| miRNA | pvalue |
|---|---|
| hsa.miR.1204 | 0.0006 |
| hsa.miR.185. | 0.0070 |
| hsa.miR.1276 | 0.0076 |
| hsa.miR.342.3p | 0.0098 |
| hsa.miR.615.3p | 0.0100 |
| hsa.miR.631 | 0.0105 |
| hsa.miR.326 | 0.0121 |
| hsa.miR.382 | 0.0128 |
| hsa.miR.374b. | 0.0128 |
| hsa.miR.601 | 0.0139 |
| hsa.miR.876.3p | 0.0158 |
| hsa.miR.488 | 0.0160 |
| hsa.miR.1261 | 0.0166 |
| hsa.miR.509.3.5p | 0.0179 |
| hsa.miR.29b.1. | 0.0201 |
| hsa.miR.520d.5p | 0.0221 |
| hsa.miR.154. | 0.0230 |
| hsa.miR.142.5p | 0.0234 |
| hsa.miR.513a.5p | 0.0261 |
| hsa.miR.29b.2. | 0.0275 |
| hsa.miR.505. | 0.0281 |
| hsa.miR.1270 | 0.0288 |
| hsa.miR.302c. | 0.0291 |
| sv40.miR.S1.5p | 0.0291 |
| hsa.miR.1255a | 0.0306 |
| hsa.miR.625. | 0.0313 |
| hsa.miR.620 | 0.0317 |
| hsa.miR.424. | 0.0337 |
| hsa.miR.542.3p | 0.0341 |
| hsa.miR.920 | 0.0353 |
| hsa.miR.551a | 0.0366 |
| hsa.miR.1301 | 0.0428 |
| hsa.miR.663 | 0.0434 |
| hsa.miR.1827 | 0.0436 |
| hsa.miR.1284 | 0.0445 |
| hsa.miR.592 | 0.0452 |
| hsa.miR.24.1. | 0.0453 |
| hsa.miR.921 | 0.0490 |
| hsa.miR.874 | 0.0496 |
| hsa.miR.675 | 0.0519 |
| hsa.miR.934 | 0.0535 |
| hsa.miR.640 | 0.0536 |
| hsa.miR.200b | 0.0558 |
| hsa.miRPlus.A1065 | 0.0562 |
| hsa_SNORD3. | 0.0579 |
| hsa.miR.136 | 0.0608 |
| hsa.miR.423.3p | 0.0632 |
| hsa.miR.548b.5p | 0.0634 |
| hsa.miR.7 | 0.0636 |
| hsa.miR.1908 | 0.0637 |

TABLE 4b
Univariate linear regression of thickness, with adjustment of stage

| miRNA | pvalue |
|---|---|
| hsa.miR.513b | 1.74E−06 |
| hsa.miR.542.3p | 6.03E−06 |
| hsa.miR.1258 | 6.90E−06 |
| hsa.miR.378 | 1.52E−05 |
| hsa.miR.628.3p | 1.90E−05 |
| hsa.miR.30c.1. | 2.36E−05 |
| hsa.miR.877 | 2.66E−05 |
| hsa.miR.516b | 3.23E−05 |
| hsa.miR.589 | 3.35E−05 |
| hsa.miR.155 | 4.30E−05 |
| hsa.miR.140.3p | 5.68E−05 |
| hsa.miR.101 | 5.82E−05 |
| hsa.miR.193b. | 7.51E−05 |
| hsa.miR.26b | 8.78E−05 |
| hsa.miR.106a | 9.45E−05 |
| hsa.miR.452 | 0.00011 |
| hsa.miR.490.5p | 0.00016 |
| hsa.miR.17 | 0.00017 |
| hsa.miR.509.5p | 0.00017 |
| hsa.miR.552 | 0.00018 |
| hsa.miR.195 | 0.00019 |
| hsa.miR.1272 | 0.00022 |
| hsa.miR.25. | 0.00028 |
| hsa.miR.215 | 0.00035 |
| hsa.miR.516a.5p | 0.00037 |
| hsa.miRPlus.C1087 | 0.00039 |
| hsa.miR.320a | 0.00041 |
| hsa.miR.320d | 0.00044 |
| hsa.miR.30b | 0.00051 |
| hsa.miR.30a | 0.00055 |
| hsa.miR.20a | 0.00057 |
| hsa.miR.125b.1. | 0.00059 |
| hsa.miR.19b | 0.00064 |
| hsa.miR.509.3.5p | 0.00067 |
| hsa.miR.320c | 0.00072 |
| hsa.miR.490.3p | 0.00075 |
| hsa.miR.130a | 0.00076 |
| hsa.miR.744 | 0.00095 |
| hsa.miR.15a | 0.00097 |
| hsa.miR.645 | 0.00102 |
| hsa.miR.1184 | 0.00109 |
| hsa.miR.320b | 0.00111 |
| hsa.miR.92a | 0.00115 |
| hsa.miR.513a.3p | 0.00130 |
| hsa.miR.20b | 0.00130 |
| hsa.miR.30c | 0.00154 |
| hsa.miR.498 | 0.00154 |
| hsa.miR.138.1. | 0.00159 |
| hsa.miR.30d | 0.00161 |
| hsa.miR.937 | 0.00167 |

TABLE 4c
Univariate Cox proportional hazard model of recurrence-
free survival, with adjustment of stage

| miRNA | pvalue |
|---|---|
| hsa.miR.1204 | 0.0003 |
| hsa.miR.374b. | 0.0006 |
| hsa.miR.382 | 0.0098 |
| hsa.miR.185. | 0.0103 |
| hsa.miR.1301 | 0.0168 |
| hsa.miR.601 | 0.0173 |
| hsa.miR.488 | 0.0177 |
| hsa.miR.542.3p | 0.0186 |
| hsa.miR.342.3p | 0.0199 |
| hsa.miR.29b.1. | 0.0218 |
| hsa.miR.1270 | 0.0241 |
| hsa.miR.142.5p | 0.0243 |
| hsa.miR.1276 | 0.0270 |
| hsa.miR.1261 | 0.0283 |
| hsa.miR.921 | 0.0285 |
| hsa.miR.505. | 0.0290 |
| hsa.miR.876.3p | 0.0293 |
| hsa.miR.24.1. | 0.0305 |
| hsa.miR.17 | 0.0312 |
| sv40.miR.S1.5p | 0.0323 |
| hsa.miR.302c. | 0.0344 |
| hsa.miR.378 | 0.0353 |
| hsa.miR.575 | 0.0367 |
| hsa.miR.920 | 0.0371 |
| hsa.miR.340. | 0.0374 |
| hsa.miR.592 | 0.0374 |
| hsa.miR.874 | 0.0403 |
| hsa.miR.520d.5p | 0.0404 |
| hsa.miR.663 | 0.0407 |
| hsa_SNORD3. | 0.0421 |
| hsa.miR.631 | 0.0424 |
| hsa.miR.106a | 0.0438 |
| hsa.miR.29b.2. | 0.0460 |
| hsa.miR.424 | 0.0464 |
| hsa.miR.1827 | 0.0470 |
| hsa.miR.20a | 0.0483 |
| hsa.miR.934 | 0.0485 |
| hsa.miR.509.3.5p | 0.0502 |
| hsa.miR.1272 | 0.0508 |
| hsa.miR.922 | 0.0517 |
| hsa.miR.513a.5p | 0.0539 |
| hsa.miR.424. | 0.0542 |
| hsa.miR.513a.3p | 0.0550 |
| hsa.miR.1284 | 0.0589 |
| hsa.miR.1908 | 0.0625 |
| hsa.miR.422a | 0.0631 |
| hsa.miR.187. | 0.0650 |
| hsa.miR.640 | 0.0657 |
| hsa.miR.1183 | 0.0675 |
| hsa.miR.326 | 0.0677 |

TABLE 4d
Univariate logistic regression of ulceration,
with adjustment of stage

| miRNA | pvalue |
|---|---|
| hsa.miR.339.5p | 0.0036 |
| hsa.miR.297 | 0.0051 |
| hsa.miR.423.3p | 0.0070 |
| hsa.miR.302e | 0.0077 |
| hsa.miR.490.3p | 0.0079 |
| hsa.miR.99b | 0.0080 |
| hsa.miR.183. | 0.0111 |
| hsa.miR.650 | 0.0120 |
| hsa.miR.876.3p | 0.0122 |
| hsa.miR.663 | 0.0131 |
| hsa.miR.125b | 0.0131 |
| hsa.miR.107 | 0.0145 |
| hsa.miR.1299 | 0.0159 |
| hsa.miR.891a | 0.0162 |
| hsa.miR.629. | 0.0163 |
| hsa.miRPlus.C1066 | 0.0212 |
| hsa.miR.205 | 0.0229 |
| hsa.miR.200c | 0.0245 |
| hsa.let.7b | 0.0255 |
| hsa.miR.200b | 0.0281 |
| hsa.miR.574.5p | 0.0291 |
| hsa.miR.548b.5p | 0.0295 |
| hsa.miR.424. | 0.0310 |
| hsa.miRPlus.A1065 | 0.0313 |
| hsa.miR.199a.5p | 0.0331 |
| hsa.miR.29b.1. | 0.0351 |
| hsa.miR.516a.5p | 0.0357 |
| hsa.miR.32. | 0.0360 |
| hsa.miR.451 | 0.0388 |
| hsa.miR.654.5p | 0.0432 |

In the discovery cohort, most surviving patients have at least 3 years follow-up; therefore almost all patients for model selection were able to be used to ensure good statistical power. However, it is arbitrary to dichotomize patients into recurrent vs non-recurrent at the 3-year mark. Five-year or 10-year recurrence could be also important endpoints for stage I/II patients. Generally, it is of interest to identify a predictive signature capable of robustly classifying patients into low vs. high risk groups corresponding to long vs. short recurrence-free survival (RFS). Towards this goal, miRNAs were ranked not only based on their univariate association with 3-year recurrence with adjustment of tumor stage (logistic model), but also with RFS (Cox PH model). In addition, miRNAs were also ranked by their association with thickness or ulceration, since it is well known that primary tumor thickness and ulceration are associated with melanoma patient RFS and overall survival. Therefore, the 339 highly expressed miRNAs were ranked according to four endpoints: 3-year recurrence (Table 4a. Univariate logistic regression of 3-year recurrence, with adjustment of stage), tumor thickness (Table 4b. Univariate linear regression of thickness, with adjustment of stage), recurrence-free survival (RFS) (Table 4c. Univariate Cox proportional hazard model of recurrence-free survival, with adjustment of stage) and ulceration (Table 4d. Univariate logistic regression of ulceration, with adjustment of stage).

Those miRNAs that are ranked high on such lists provided initial candidates for predictors in selecting multivariate models to predict RFS.

Starting from the top ranked miRNAs in each list in Tables 4a-4d, multivariate logistic regression models were constructed via upward and downward model selection to maximize area under the receiver operating characteristic (ROC) curve with 4-fold cross-validation. Therefore, starting from top ranked miRNAs in the first list (Table 4a), the following Model 1 was selected by maximizing area under the receiver operating characteristic (ROC) curve for 3-year recurrence with 4-fold cross-validation. Note that, within Model 1, hsa-miR-1204, hsa-miR-342-3p, hsa-miR-374b* and hsa-miR-625* are among top 30 of Table 4a. hsa-miR-516b is among top 10 of Table 4b. Model 2 was similarly selected starting from the top ranked miRNAs in Table 4a, by maximizing AUC with cross validation. Note that, within Model 2, hsa-miR-1204, hsa-miR-342-3p and hsa-miR-374b* are among top 10 of Table 4b. hsa-miR-663 and SNORD3A are among top 30 of Table 4c. Model 3 was selected starting from the top ranked miRNAs in Table 4b, by maximizing AUC with cross validation. Note that, within Model 3, hsa-miR-513b is top 1 and hsa-miR-215 is top 24 in Table 4b. hsa-miR-615-3p and hsa-miR-154* are among top 20 of Table 4a. Model 4 was selected starting from top ranked miRNAs in Table 4c, by maximizing AUC with cross validation. Note that, within Table 4(d) the miRNAs hsa-miR-1204, hsa-miR-374b*, hsa-miR-382 and hsa-miR-1276 are among top 15 of Table 4(c)). Each of the four models achieved an area under the ROC between 94% and 96% in the discovery cohort and between 84% and 96% in the validation cohort. Some top-ranked miRNAs remained significant in the selected predictive models while others were replaced by multivariately-significant predictive miRNAs not ranked so high among the four univariately-ranked lists. Given the limited sample size in the discovery stage, we would like to avoid eliminating potentially high-value miRNAs prematurely, thus 4 logistic regression models were selected with a combined total of 21 miRNAs, as the predictive signature set. Each of the four models achieved an area under the ROC between 94% and 96% in the discovery cohort. The risk scores from the four models were averaged to form the final classifier which was discussed earlier. The models (FIG. 17) were then used to calculate the coefficients which therefore can be used to calculate different linear combinations of the signature sets (Tables 5a-d). The details of the four models are as follows:

TABLE 5a
Coefficients and p-values of Model 1

| | p | coefficient |
|---|---|---|
| stage | 0.311 | 1.053 |
| hsa-miR-1204 | 0.004 | 9.723 |
| hsa-miR-342-3p | 0.024 | −1.411 |
| hsa-miR-374b* | 0.006 | −8.010 |
| hsa-miR-516b | 0.056 | 3.126 |
| hsa-miR-625* | 0.053 | 2.724 |
| hsa-miR-1285 | 0.009 | 5.075 |
| hsa-let-7a-2* | 0.010 | −8.446 |
| hsa-miR-564 | 0.013 | 3.621 |
| hsa-miR-574-3p | 0.095 | 3.106 |

TABLE 5b
Coefficients and p-values of Model 2

| | p | coefficient |
|---|---|---|
| stage | 0.726 | 0.308 |
| hsa-miR-1204 | 0.001 | 11.652 |
| hsa-miR-342-3p | 0.003 | −2.035 |
| hsa-miR-374b* | 0.001 | −9.366 |
| hsa-miR-663 | 0.005 | −5.029 |
| SNORD3A | 0.016 | 1.716 |
| hsa-miR-564 | 0.040 | 2.714 |
| hsa-miR-10a | 0.005 | −2.064 |

TABLE 5c
Coefficients and p-values of Model 3

| | p | coefficient |
|---|---|---|
| stage | 0.466 | 0.917 |
| hsa-miR-513b | 0.085 | 4.113 |
| hsa-miR-215 | 0.089 | −4.562 |
| hsa-miR-7 | 0.097 | 6.749 |
| hsa-miR-374b* | 0.012 | −20.952 |
| hsa-miR-615-3p | 0.014 | 7.655 |
| hsa-miR-154* | 0.035 | 7.899 |
| hsa-miR-377* | 0.012 | 8.782 |
| hsa-miR-1285 | 0.007 | 7.171 |
| hsa-miR-574-3p | 0.018 | 7.205 |
| hsa-let-7a-2* | 0.013 | −15.125 |
| hsa-miR-564 | 0.016 | 4.978 |

TABLE 5d
Coefficients and p-values of Model 4

| | p | coefficient |
|---|---|---|
| stage | 0.094 | 2.039 |
| hsa-miR-1204 | 0.032 | 11.581 |
| hsa-miR-374b* | 0.009 | −13.646 |
| hsa-miR-382 | 0.044 | 2.885 |
| hsa-miR-1276 | 0.007 | 4.951 |
| hsa-miR-615-3p | 0.027 | 7.046 |
| hsa-miR-377* | 0.018 | 7.770 |
| hsa-miR-574-3p | 0.057 | 3.320 |
| hsa-miR-1285 | 0.019 | 5.006 |
| hsa-let-7a-2* | 0.004 | −9.968 |
| hsa-miR-99b | 0.076 | 5.360 |

### Example 9

### miRNAs hsa-miR-215, hsa-miR-374b*, hsa-miR-382, hsa-miR-516b, and hsa-miR-7 are Shown to be Suppressors of Metastasis

The data herein support that a paradigm of combining the 1) identification of molecular alterations from large datasets generated from human tissue with 2) a functional screening platform is a more robust way to filter important events in tumorigenesis than either one alone. As such, from this set of differentially expressed miRNAs, 40 candidates were screened in an automated in vitro invasion assay, with careful control of cell proliferation effects, to identify potential metastasis modulators. A panel of cell lines was tested to ensure effects were applicable to most, if not all, melanomas. The analyses identified five miRNAs (hsa-miR-215, hsa-miR-374b*, hsa-miR-382, hsa-miR-516b, and hsa-miR-7) that consistently repressed invasion. Of the five miRNAs identified, it was observed that three (miR-382, miR-516b, and miR-7) are suppressors of metastasis in vivo. Further, analysis of the clinical data shows that miR-374b*, miR-382, miR-516b expression independently correlates with overall survival of these patients, highlighting their importance in melanoma progression (FIG. 15).

The miRNAs identified have lower expression in aggressive primary tumors; thus in order to more closely recapitulate what occurs in the primary tumor, miR-382, miR-516b, and miR-7 were inhibited in a poorly invasive cell line to probe for effects on invasion Inhibition of miR-382 and miR-516b alone or in combination enhanced the invasive capacity of these cells, further supporting the biological relevance of the findings (FIG. 16).

### REFERENCES

1. Tas F, Mudun A, Kirma C. Cardiac involvement in melanoma: a case report and review of the literature. J Cancer Res Ther., 2010; 6(3):359-61.
2. Valcárcel et al., Vascular endothelial growth factor regulates melanoma cell adhesion and growth in the bone marrow microenvironment via tumor cyclooxygenase-2. J Transl Med. 2011; 9:142.
3. Di Cesare S, Maloney S, Fernandes B F, Martins C, Marshall J C, Antecka E, Odashiro A N, Dawson W W, Burnier M N The effect of blue light exposure in an ocular melanoma animal model. J Exp Clin Cancer Res. 2009; 28:48.
4. Tsao et al., Management of cutaneous melanoma. N Engl J Med 2004; 351:998-1012.
5. Piris, A., Mihm, M. & Duncan, L. AJCC melanoma staging update: impact on dermatopathology practice and patient management. Journal of Cutaneous Pathology, 2011, 38, 394-400.
6. Balch, C., et al. Final version of 2009 AJCC melanoma staging and classification. Journal of Clinical Oncology: official journal of the American Society of Clinical Oncology 27, 6199-6405 (2009).
7. Azzola, M., et al. Tumor mitotic rate is a more powerful prognostic indicator than ulceration in patients with primary cutaneous melanoma: an analysis of 3661 patients from a single center. Cancer 97, 1488-1586 (2003).
8. Schmid-Wendtner, M., et al. Prognostic index for cutaneous melanoma: an analysis after follow-up of 2715 patients. Melanoma Research 11, 619-645 (2001).
9. Leiter, U., et al. Hazard rates for recurrent and secondary cutaneous melanoma: An analysis of 33,384 patients in the German Central Malignant Melanoma Registry. Journal of the American Academy of Dermatology, 2011, 66, 37-82.
10. Balch C M, Gershenwald J E, Soong S J, Thompson J F, Atkins M B, Byrd D R, Buzaid A C, Cochran A J, Coit D G, Ding S, Eggermont A M, Flaherty K T, Gimotty P A, Kirkwood J M, McMasters K M, Mihm M C Jr, Morton D L, Ross M I, Sober A J, Sondak V K. Final version of 2009 AJCC melanoma staging and classification. J Clin Oncol. 2009; 27(36):6199-206.
11. Berger A J, Camp R L, Divito K A, Kluger H M, Halaban R, Rimm D L. Automated quantitative analysis of HDM2 expression in malignant melanoma shows association with early-stage disease and improved outcome. Cancer Res 2004; 64:8767-72.
12. O'Reilly K E, Rojo F, She Q B, et al. mTOR inhibition induces upstream receptor tyrosine kinase signaling and activates Akt. Cancer Res 2006; 66:1500-8.
13. Yancovitz M, Yoon J, Mikhail M, et al. Detection of mutant BRAF alleles in the plasma of patients with metastatic melanoma. J Mol Diagn 2007; 9:178-83.
15. Mansfield A S, Markovic S N. Novel therapeutics for the treatment of metastatic melanoma. Future Oncol. 2009; 5:543-557.
16. Palmer S R, Erickson L A, Ichetovkin I, Knauer D J, Markovic S N. Circulating serologic and molecular biomarkers in malignant melanoma. Mayo Clin Proc. 2011; 86(10):981-90.
17. Perou, C., et al. Molecular portraits of human breast tumours. Nature 406, 747-799 (2000).
18. Glinsky, G., Glinskii, A., Stephenson, A., Hoffman, R. & Gerald, W. Gene expression profiling predicts clinical outcome of prostate cancer. The Journal of Clinical Investigation 113, 913-936 (2004).
19. van 't Veer, L., et al. Gene expression profiling predicts clinical outcome of breast cancer. Nature 415, 530-536 (2002).
20. Paik, S., et al. A multigene assay to predict recurrence of tamoxifen-treated, node-negative breast cancer. The New England journal of medicine 351, 2817-2843 (2004).
21. Johnson, S., et al. RAS is regulated by the let-7 microRNA family. Cell 120, 635-682 (2005).
22. Turajlic, S., et al. Whole genome sequencing of matched primary and metastatic acral melanomas. Genome Res. 2012; 22(2): 196-207.
23. Segura, M., et al. Melanoma MicroRNA signature predicts post-recurrence survival. Clinical Cancer Research: an official journal of the American Association for Cancer Research, 2010, 16, 1577-1663.
24. Gaziel-Sovran, A., et al. miR-30b/30d regulation of GalNAc transferases enhances invasion and immunosuppression during metastasis. Cancer Cell, 2011, 20, 104-122.
25. Levy, C., et al. Intronic miR-211 assumes the tumor suppressive function of its host gene in melanoma. Mol Cell, 2010, 40, 841-849.
26. Png, K., et al. MicroRNA-335 inhibits tumor reinitiation and is silenced through genetic and epigenetic mechanisms in human breast cancer. Genes & Development, 2011, 25, 226-257.
27. Png, K. J., Halberg, N., Yoshida, M. & Tavazoie, S. F. A microRNA regulon that mediates endothelial recruitment and metastasis by cancer cells. Nature, 2011; 481(7380): 190-194
28. Segura, M., et al. Aberrant miR-182 expression promotes melanoma metastasis by repressing FOXO3 and microphthalmia-associated transcription factor. Proceedings of the National Academy of Sciences of the United States of America 106, 1814-1823 (2009).
29. Tavazoie, S., et al. Endogenous human microRNAs that suppress breast cancer metastasis. Nature 451, 147-199 (2008).
30. Johnson, S., et al. RAS is regulated by the let-7 microRNA family. Cell 120, 635-682 (2005).
31. Rabinowits et al. Clin Lung Cancer, 2009, 10:42-46.
32. Sambrook, Fritsch & Maniatis, Molecular Cloning: A Laboratory Manual, Second Edition. Cold Spring Harbor, N.Y.: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.).
33. Current Protocols in Molecular Biology. John Wiley & Sons, Inc., 1994.
34. Kunkel, Proc. Natl. Acad. Sci. USA 82: 488-492 (1985).
35. U.S. Pat. No. 5,071,743.
36. Fukuoka et al., Biochem. Biophys. Res. Commun. 263: 357-360 (1999).
37. Kim and Maas, BioTech. 28: 196-198 (2000).
38. Parikh and Guengerich, BioTech. 24: 428-431 (1998).
39. Ray and Nickoloff, BioTech. 13: 342-346 (1992).
40. Wang et al., BioTech. 19: 556-559 (1995).
41. Wang and Malcolm, BioTech. 26: 680-682 (1999).
42. Xu and Gong, BioTech. 26: 639-641 (1999).
43. U.S. Pat. No. 5,789,166
44. U.S. Pat. No. 5,932,419
45. Hogrefe, Strategies 14. 3: 74-75 (2001)
46. U.S. Pat. No. 5,702,931
47. U.S. Pat. No. 5,780,270
48. U.S. Pat. No. 6,242,222
49. Angag and Schutz, Biotech. 30: 486-488 (2001).
50. Wang and Wilkinson, Biotech. 29: 976-978 (2000).
51. Kang et al., Biotech. 20: 44-46 (1996).
52. Ogel and McPherson, Protein Engineer. 5: 467-468 (1992).
53. Kirsch and Joly, Nucl. Acids. Res. 26: 1848-1850 (1998).
54. Rhem and Hancock, J. Bacteriol. 178: 3346-3349 (1996).
55. Boles and Miogsa, Curr. Genet. 28: 197-198 (1995).
56. Barrenttino et al., Nuc. Acids. Res. 22: 541-542 (1993).
57. Tessier and Thomas, Meths. Molec. Biol., 1996, 57: 229-237.
58. Pons et al., Meth. Molec. Biol., 1997, 67: 209-218.

LIST OF SEQUENCES:

| | | SEQ |
|---|---|---|
| | | ID |
| NAME | SEQUENCE | NO: |
| hsa-miR- | UACCCUGUAGAUCCGAAUUUGUG | 1 |
| 10a | | |
| hsa-miR- | UCUGGGCAACAAAGUGAGACCU | 2 |
| 1285 | | |
| hsa-miR- | CUUAGCAGGUUGUAUUAUCAUU | 3 |
| 374b* | | |
| hsa-miR- | AGAGGUUGCCCUUGGUGAAUUC | 4 |
| 377* | | |
| hsa-miR- | UUCACAAGGAGGUGUCAUUUAU | 5 |
| 513b | | |
| hsa-miR- | UCUCACACAGAAAUCGCACCCGU | 6 |
| 342-3p | | |
| hsa-miR- | GACUAUAGAACUUUCCCCCUCA | 7 |
| 625* | | |
| SNORD3A | AAGACTATACTTTCAGGGATCATTTCTATAGTGT | 8 |
| | GTTACTAGAGAAGTTTCTCTGAACGTGTAGAGC | |
| | ACCGAAAACCACGAGGAAGAGAGGTAGCGTTTT | |
| | CTCCTGAGCGTGAAGCCGGCTTTCTGGCGTTGCT | |
| | TGGCTGCAACTGCCGTCAGCCATTGATGATCGTT | |
| | CTTCTCTCCGTATTGGGGAGTGAGAGGGAGAGA | |
| | ACGCGGTCTGAGTGGT | |
| hsa-miR- | UCGUGGCCUGGUCUCCAUUAU | 9 |
| 1204 | | |
| hsa-miR- | CACGCUCAUGCACACACCCACA | 10 |
| 574-3p | | |
| hsa-let- | CUGUACAGCCUCCUAGCUUUCC | 11 |
| 7a-2* | | |
| hsa-miR- | UCCGAGCCUGGGUCUCCCUCUU | 12 |
| 615-3p | | |
| hsa-miR- | AGGCACGGUGUCAGCAGGC | 13 |
| 564 | | |
| hsa-miR- | AAUCAUACACGGUUGACCUAUU | 14 |
| 154* | | |
| hsa-miR- | UGGAAGACUAGUGAUUUUGUUGU | 15 |
| 7 | | |
| hsa-miR- | AUGACCUAUGAAUUGACAGAC | 16 |
| 215 | | |
| hsa-miR- | GAAGUUGUUCGUGGUGGAUUCG | 17 |
| 382 | | |
| hsa-miR- | AGGCGGGGCGCCGCGGGACCGC | 18 |
| 663 | | |
| hsa-miR- | AUCUGGAGGUAAGAAGCACUUU | 19 |
| 516b | | |
| hsa-miR- | CACCCGUAGAACCGACCUUGCG | 20 |
| 99b | | |
| hsa-miR- | UAAAGAGCCCUGUGGAGACA | 21 |
| 1276 | | |
| primer | TAGGCGATCGCTCGAGCCAAGCCAACATCTCCA | 22 |
| Fwd | GAC | |
| primer | AATTCCCGGGCTCGAGGATGTTGTCATTGCTTTA | 23 |
| Rvs | TTACTCA | |
| SCR#1 | UCACAACCUCCUAGAAAGAGUAGA | 24 |
| SCR#2 | UUGUACUACACAAAAGUACUG | 25 |

Source of miR and SNORD3A sequences: http://www.mirbase.org/

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method for predicting the likelihood of recurrence of melanoma in a subject diagnosed with melanoma, said method comprising:
a. measuring the levels of four or more miRNAs selected from the group consisting of miR-10a, miR-1285, miR-377*, miR-513b, miR-342-3p, miR-625*, SNORD3A, miR-1204, miR-574-3p, let-7a-2*, miR-615-3p, miR-564, miR-154*, miR-663, miR-99b, miR-1276, miR-215, miR-374b*, miR-382, miR-516b, and miR-7, in a melanoma sample collected from the subject;
b. calculating combined levels of the miRNAs measured in step (a);
c. comparing the combined levels of the miRNAs measured in step (a) with the corresponding combined control levels of said miRNAs, and
d. (i) identifying the subject as being at high risk of melanoma recurrence if the combined levels of the miRNAs measured in step (a) are higher than the corresponding combined control levels or (ii) identifying the subject as being at low risk of melanoma recurrence if the combined levels of the miRNAs measured in step (a) are same or lower than the corresponding combined control levels.

2-7. (canceled)

8. The method of claim 1, wherein the combined control levels are a predetermined standard.

9. The method of claim 1, wherein the combined control levels are the combined levels of the same miRNAs in a non-recurrent melanoma sample.

10. The method of claim 1, further comprising administering to the subject determined as being at high risk of melanoma recurrence a melanoma treatment.

11. The method of claim 10, wherein the melanoma treatment is selected from the group consisting of Interleukin 2 (IL2), Aldesleukin (Proleukin), Dacarbazine (DTIC-Dome), Ipilimumab (Yervoy), temozolomide, Vemurafenib (Zelboraf), and any combinations thereof.

12. The method of claim 1, wherein the subject is human.

13. The method of claim 1, wherein the subject is an experimental animal.

14. The method of claim 1, which method comprises a step of collecting the melanoma sample from the subject.

15. The method of claim 1, wherein the levels of the miRNAs are determined using a method selected from the group consisting of hybridization, RT-PCR, and sequencing.

16. The method of claim 1, wherein, prior to measuring miRNA level, the miRNA is purified from the melanoma sample.

17. The method of claim 1, further comprising the step of reducing or eliminating degradation of the miRNAs.

18. The method of claim 1, further comprising recruiting the subject in a clinical trial.

19. A kit comprising primers or probes specific for four or more miRNAs selected from the group consisting of miR-10a, miR-1285, miR-374b *, miR-377*, miR-513b, miR-342-3p, miR-625*, SNORD3A, miR-1204, miR-574-3p, let-7a-2*, miR-615-3p, miR-564, miR-154*, miR-7, miR-215, miR-382, miR-663, miR-516b, miR-99b, and miR-1276.

20-25. (canceled)

26. The kit of claim 19, further comprising miRNA isolation or purification means.

27. The kit of claim 19, further comprising instructions for use.

28. A method for treatment of a melanoma recurrence in a subject in need thereof comprising increasing the level and/or activity of at least one miRNA selected from the group consisting of miR-215, miR-374b*, miR-382, miR-516b, and miR-7 in the melanoma cells of the subject.

29. The method of claim 1, wherein the melanoma recurrence is metastasis.

30. The method of claim 28, wherein the melanoma recurrence is metastasis.


SEQUENCE LISTING (25 sequences)

SEQ ID NO: 1 - RNA, 23 nt, Homo sapiens
uacccuguagauccgaauuugug

SEQ ID NO: 2 - RNA, 22 nt, Homospaiens
ucugggcaacaaagugagaccu

SEQ ID NO: 3 - RNA, 22 nt, Homo sapiens
cuuagcagguuguauuaucauu

SEQ ID NO: 4 - RNA, 22 nt, Homo sapiens
agagguugcccuuggugaauuc

SEQ ID NO: 5 - RNA, 22 nt, Homo sapiens
uucacaaggaggugucauuuau

SEQ ID NO: 6 - RNA, 23 nt, Homo sapiens
ucucacacagaaaucgcacccgu

SEQ ID NO: 7 - RNA, 22 nt, Homo sapiens
gacuauagaacuuucccccuca

SEQ ID NO: 8 - DNA, 217 bp, Homo sapiens
aagactatactttcagggatcatttctatagtgtgttactagagaagtttctctgaacgtgtagagcaccgaaaaccacgaggaagagaggtagcgttttctcctgagcgtgaagccggctttctggcgttgcttggctgcaactgccgtcagccattgatgatcgttcttctctccgtattggggagtgagagggagagaacgcggtctgagtggt

SEQ ID NO: 9 - RNA, 21 nt, Homo sapiens
ucguggccuggucuccauuau

SEQ ID NO: 10 - RNA, 22 nt, Homo sapiens
cacgcucaugcacacacccaca

SEQ ID NO: 11 - RNA, 22 nt, Homo sapiens
cuguacagccuccuagcuuucc

SEQ ID NO: 12 - RNA, 22 nt, Homo sapiens
uccgagccugggucucccucuu

SEQ ID NO: 13 - RNA, 19 nt, Homo sapiens
aggcacggugucagcaggc

SEQ ID NO: 14 - RNA, 22 nt, Homo sapiens
aaucauacacgguugaccuauu

SEQ ID NO: 15 - RNA, 23 nt, Homo sapiens
uggaagacuagugauuuuguugu

SEQ ID NO: 16 - RNA, 21 nt, Homo sapiens
augaccuaugaauugacagac

SEQ ID NO: 17 - RNA, 22 nt, Homo sapiens
gaaguuguucgugguggauucg

SEQ ID NO: 18 - RNA, 22 nt, Homo sapiens
aggcggggcgccgcgggaccgc

SEQ ID NO: 19 - RNA, 22 nt, Homo sapiens
aucuggagguaagaagcacuuu

SEQ ID NO: 20 - RNA, 22 nt, Homo sapiens
cacccguagaaccgaccuugcg

SEQ ID NO: 21 - RNA, 20 nt, Homo sapiens
uaaagagcccuguggagaca

SEQ ID NO: 22 - DNA, 36 bp, Artificial Sequence
  Description of Artificial Sequence Synthetic primer
taggcgatcgctcgagccaagccaacatctccagac

SEQ ID NO: 23 - DNA, 41 bp, Artificial Sequence
  Description of Artificial Sequence Synthetic primer
aattcccgggctcgaggatgttgtcattgctttattactca

SEQ ID NO: 24 - RNA, 24 nt, Artificial Sequence
  Description of Artificial Sequence Synthetic oligonucleotide
ucacaaccuccuagaaagaguaga

SEQ ID NO: 25 - RNA, 21 nt, Artificial Sequence
  Description of Artificial Sequence Synthetic oligonucleotide
uuguacuacacaaaaguacug